SEQUENCE LISTING (45 sequences)

SEQ ID NO: 1 - skipped

SEQ ID NO: 2 - protein, 5 aa, synthetic construct
  REGION 1..5: note = Synthetic
RAYAR

SEQ ID NO: 3 - protein, 5 aa, synthetic construct
  REGION 1..5: note = Synthetic
KAYAK

SEQ ID NO: 4-5 - skipped (2 entries)

SEQ ID NO: 6 - protein, 29 aa, synthetic construct
  REGION 1..29: note = Synthetic
QARILEADAEILRAYARILEAHAEILRAQ

SEQ ID NO: 7 - protein, 29 aa, synthetic construct
  REGION 1..29: note = Synthetic
QAKILEADAEILKAYAKILEAHAEILKAQ

SEQ ID NO: 8 - protein, 23 aa, synthetic construct
  REGION 1..23: note = Synthetic
ADAEILRAYARILEAHAEILRAQ

SEQ ID NO: 9 - protein, 10 aa, synthetic construct
  REPEAT 2..10: note = may or may not be present
GGGGGGGGGG

SEQ ID NO: 10 - protein, 4 aa, synthetic construct
  REGION 1..4: note = Synthetic
SGSG

SEQ ID NO: 11 - protein, 4 aa, synthetic construct
  REGION 1..4: note = Synthetic
GSGS

SEQ ID NO: 12 - protein, 4 aa, synthetic construct
  REGION 1..4: note = Synthetic
SSSS

SEQ ID NO: 13 - protein, 4 aa, synthetic construct
  REGION 1..4: note = Synthetic
GGGS

SEQ ID NO: 14 - skipped

SEQ ID NO: 15 - protein, 24 aa, synthetic construct
GGCGGCGGCGGCGGCGGCGGCGGC

SEQ ID NO: 16 - protein, 15 aa, synthetic construct
GGGGSGGGGSGGGGS

SEQ ID NO: 17 - protein, 13 aa, synthetic construct
  REGION 1..13: note = Synthetic
LEEKKGNYVVTDH

SEQ ID NO: 18 - protein, 12 aa, synthetic construct
  REGION 1..12: note = Synthetic
  SITE 1: note = MISC_FEATURE - D-alanine
  SITE 3: note = MISC_FEATURE - cyclohexylalanine
  SITE 12: note = MISC_FEATURE - D-alanine
XKXVAAWTLKAX

SEQ ID NO: 19 - protein, 33 aa, synthetic construct
  REGION 1..33: note = Synthetic
SGSGQARILEADAEILRAYARILEAHAEILRAQ

SEQ ID NO: 20 - protein, 46 aa, synthetic construct
  REGION 1..46: note = Synthetic
LEEKKGNYVVTDHSGSGQARILEADAEILRAYARILEAHAEILRAQ

SEQ ID NO: 21 - protein, 45 aa, synthetic construct
  SITE 1: note = MISC_FEATURE - D-alanine
  SITE 12: note = MISC_FEATURE - D-alanine
  SITE 3: note = MISC_FEATURE - cyclohexylalanine
XKXVAAWTLKAXSGSGQARILEADAEILRAYARILEAHAEILRAQ

SEQ ID NO: 22 - protein, 17 aa, synthetic construct
  REGION 1..17: note = Synthetic
ISQAVHAAHAEINEAGR

SEQ ID NO: 23 - protein, 8 aa, synthetic construct
  REGION 1..8: note = Synthetic
SIINFEKL

SEQ ID NO: 24 - protein, 5 aa, synthetic construct
  REGION 1..5: note = Synthetic
AAYGG

SEQ ID NO: 25 - protein, 12 aa, synthetic construct
  REGION 1..12: note = Synthetic
NPSHPLSGGGGS

SEQ ID NO: 26 - protein, 13 aa, synthetic construct
  REGION 1..13: note = Synthetic
NPSHPLSGGGGSK

SEQ ID NO: 27 - protein, 14 aa, synthetic construct
  REGION 1..13: note = Synthetic
NPSHPLSGGGGSKK

SEQ ID NO: 28 - protein, 9 aa, synthetic construct
  REGION 1..9: note = Synthetic
AVGIGAVFL

SEQ ID NO: 29 - protein, 5 aa, synthetic construct
  REGION 1..5: note = Synthetic
KSGSG

SEQ ID NO: 30 - protein, 6 aa, synthetic construct
  REGION 1..6: note = Synthetic
KKSGSG

SEQ ID NO: 31 - protein, 5 aa, synthetic construct
  REGION 1..5: note = Synthetic
  REPEAT 1..5: note = repeat unit:2 times
EAAAK

SEQ ID NO: 32 - protein, 29 aa, synthetic construct
  REGION 1..29: note = Synthetic
QSRITEADSEITRAYSRITEAHSEITRAQ

SEQ ID NO: 33 - protein, 29 aa, synthetic construct
  REGION 1..29: note = Synthetic
QARILEADAEILRAYAEILEAHAEILRAQ

SEQ ID NO: 34 - protein, 29 aa, synthetic construct
QARTLESDAETLRSYARTLESHAETLRSQ

SEQ ID NO: 35 - protein, 29 aa, synthetic construct
  REGION 1..29: note = Synthetic
QARILEADAEILRAYARILEAHAEILRAQ

SEQ ID NO: 36 - protein, 29 aa, synthetic construct
  REGION 1..29: note = Synthetic
QAKILEADAEILKAYAKILEAHAEILKAQ

SEQ ID NO: 37 - protein, 23 aa, synthetic construct
  REGION 1..23: note = Synthetic
ADAEILRAYARILEAHAEILRAQ

SEQ ID NO: 38 - protein, 56 aa, synthetic construct
  REGION 1..56: note = Synthetic
AVGIGAVFLSGSGEAAAKEAAAKSGSGQARILEADAEILRAYARILEAHAEILRAQ

SEQ ID NO: 39 - protein, 47 aa, synthetic construct
  REGION 1..47: note = Synthetic
SGSGEAAAKEAAAKSGSGQARILEADAEILRAYARILEAHAEILRAQ

SEQ ID NO: 40 - protein, 13 aa, synthetic construct
SGSGAVGIGAVFL

SEQ ID NO: 41 - protein, 28 aa, synthetic construct
  REGION 1..27: note = Synthetic
AVGIGAVFLSGSGEAAAKEAAAKSGSGC

SEQ ID NO: 42 - protein, 29 aa, synthetic construct
  REGION 1..29: note = Synthetic
QSRITEADSEITRAYSRITEAHSEITRAQ

SEQ ID NO: 43 - protein, 29 aa, synthetic construct
  REGION 1..29: note = Synthetic
QARILEADAEILRAYAEILEAHAEILRAQ

SEQ ID NO: 44 - protein, 29 aa, synthetic construct
  REGION 1..29: note = Synthetic
QARTLESDAETLRSYARTLESHAETLRSQ

SEQ ID NO: 45 - protein, 12 aa, synthetic construct
  REGION 1..12: note = Synthetic
IPVLDENGLFAP

(19) United States
(12) Patent Application Publication
(10) Pub. No.: US 2025/0276058 A1
(43) Pub. Date: Sep. 4, 2025

(54) NANOFIBERS TO PRIME ANTIBODY RESPONSES AND METHODS OF USING SAME

(71) Applicant: Duke University, Durham, NC (US)

(72) Inventors: Chelsea Fries, Durham, NC (US); Genevieve Fouda, Durham, NC (US); Joel Collier, Durham, NC (US)

(21) Appl. No.: 18/693,805

(22) PCT Filed: Sep. 20, 2022
(86) PCT No.: PCT/US2022/044143

§ 371 Date: Mar. 20, 2024

Related U.S. Application Data
(60) Provisional application No. 63/245,968, filed on Sep. 20, 2021.

(51) Int. Cl. A61K 39/385; A61K 39/00; A61K 39/21; A61P 31/18; C12N 7/00

(52) U.S. Cl. CPC A61K 39/385; A61K 39/00114; A61K 39/001141; A61K 39/21; A61P 31/18; C12N 7/00; A61K 2039/575; A61K 2039/6031; C12N 2740/16034

(57) ABSTRACT

Embodiments are directed to nanofiber compositions comprising antigens, such as the fusion peptide (FP) of HIV-1 trimers. The compositions may be used as vaccines themselves, or they may be used in combination with vaccines to enhance the immune response and increase antibody titers.

### CROSS-REFERENCE TO RELATED APPLICATIONS

This patent application is the U.S. national stage entry, under 35 U.S.C. § 371, of International Application Number PCT/US2022/044143, filed Sep. 20, 2022, which claims priority to U.S. Provisional Patent Application No. 63/245,968, filed Sep. 20, 2021 the entire contents of each of which are hereby incorporated by reference.

### STATEMENT REGARDING FEDERALLY SPONSORED RESEARCH

This invention was made with government support under grant R01 AI145016 awarded by the National Institutes of Health. The government has certain rights in the invention.

### SEQUENCE LISTING

The contents of the electronic sequence listing (028193-9405-US02_Sequence_Listing.xml; Size: 53,760 bytes; and Date of Creation: Sep. 27, 2024) is herein incorporated by reference in its entirety.

### FIELD

Embodiments of this invention are directed generally to biology, medicine, and immunology. Certain aspects are directed to immunogenic fibrils and their use in inducing an immune response.

### INTRODUCTION

Broadly neutralizing antibodies (bNabs) to HIV-1 most commonly target non-contiguous conformational epitopes that often include glycosylation sites, which has made selection of peptide epitopes for HIV vaccination challenging. However, the fusion peptide (FP) of HIV-1 trimers was recently discovered as a binding site of the bNab VRC34.01. FP is a relatively conserved 8-10 amino acid sequence at the trimer's N-terminus which can rotate and twist, assuming diverse conformations which allow HIV virions to infect host cells. The identification of this short, linear epitope provides an opportunity to generate peptide immunogens which may generate protective antibody responses against HIV-1. However, immunization against FP often results in low-magnitude and low-affinity responses when it is displayed on scaffold proteins. The most effective method for inducing FP-directed antibody responses to date has been to prime the immune system with the carrier protein Keyhole Limpet Hemocyanin decorate with FP peptides, followed by boosting with native-like trimer proteins to mature antibodies towards the conformation of FP native to HIV-1 virions. This type of heterologous immunization regimen has produced single antibodies with broad, although low-potency, neutralization potential. However, the seroprevalence of these antibodies is relatively low and does not produce protective serological responses. For these reasons, improvements are needed in FP-directed antibody responses to impact the therapeutic generation of antibody responses to HIV-1.

A key challenge in the induction of high seroprevalence FP-directed antibodies after heterologous immunization is the vast number of immunodominant epitopes on HIV trimers. Even after FP-priming, antibody specificity drifts from FP specificity toward other non-neutralizing epitopes on SOSIP trimers after the multiple SOSIP boosts required for affinity maturation of FP antibodies are delivered. SOSIP is in reference to the engineered disulfide bonds between gp120 and gp41 of HIV and the isoleucine to proline mutation in the HR1 helix, which act to stabilize the pre-fusion conformation, while 664 is the final residue in truncated ectodomain. Systematic studies of germinal center dynamics have noted that increasing the number and affinity of precursor B cells within germinal centers can improve their competitive fitness upon boosting with antigens bearing multiple epitopes with varying levels of immunodominance. Improving the number and affinity of B cell clones with FP-priming immunogens may improve the fitness of these cells upon SOSIP boosting and increase the resultant seroprevalence of FP-directed antibodies.

Another emergent feature of discovered FP-directed bNabs is their ability to bind FP in disparate conformations. On native HIV trimers, FP assumes many different conformations, and antibodies which are most likely to mature to a broadly neutralizing phenotype are capable of binding multiple conformations of FP. In fact, two isolated bNabs, VRC34.01 and PGT151, bind FP in extended orientations which are rotated nearly 90° from one another, while other neutralizing antibodies bind FP in U-shaped conformations. Thus, priming immunogens which can produce antibody repertoires which bind multiple conformations of FP may be more likely to produce protective FP-directed responses. Currently, there is not a clear consensus on what features of an immunogen increase the conformational tolerance of antibodies.

### SUMMARY

In an aspect, provided herein is a composition comprising: (i) a nanofiber comprising: a backbone comprising a plurality of self-assembling peptides, wherein each self-assembling peptide forms an alpha-helix, and wherein the self-assembling peptide comprises an amino acid sequence of bXXXb (SEQ ID NO: 1), wherein X is independently any amino acid, and b is independently any positively charged amino acid; at least one glycomimetic peptide linked to the backbone; and at least one antigen, wherein each antigen is individually linked to the backbone by a helical linker; or (ii) a nanofiber comprising: a backbone comprising a plurality of self-assembling peptides, wherein each self-assembling peptide forms an alpha-helix, and wherein the self-assembling peptide comprises an amino acid sequence of bXXXb (SEQ ID NO: 1), wherein X is independently any amino acid, and b is independently any positively charged amino acid; and at least one antigen, wherein each antigen is individually linked to the backbone by a helical linker; or (iii) a nanofiber comprising: a backbone comprising a plurality of self-assembling peptides, wherein each self-assembling peptide forms an alpha-helix, and wherein the self-assembling peptide comprises an amino acid sequence of bXXXb (SEQ ID NO: 1), wherein X is independently any amino acid, and b is independently any positively charged amino acid; and at least one glycomimetic peptide linked to the backbone; or (iv) any combination of (i), (ii), and (iii). In some embodiments, the antigen comprises an epitope for a disease selected from HIV, inflammatory bowel disease, gonorrhea, or rheumatoid arthritis.

In some embodiments, bXXXb (SEQ ID NO: 1) is RAYAR (SEQ ID NO: 2) or KAYAK (SEQ ID NO: 3). In some embodiments, the backbone comprises an amino acid sequence of ZnbXXXbZm (SEQ ID NO: 5), wherein b is independently any positively charged amino acid, Z is independently any amino acid, X is independently any amino acid, n is an integer from 0 to 20, and m is an integer from 0 to 20. In some embodiments, the backbone comprises an amino acid sequence selected from QARILEADAEILRAYARILEAHAEILRAQ (SEQ ID NO: 6), or QAKILEADAEILKAYAKILEAHAEILKAQ (SEQ ID NO: 7), or ADAEILRAYARILEAHAEILRAQ (SEQ ID NO: 8), or SEQ ID NO: 35, or SEQ ID NO: 36, or SEQ ID NO: 37. In some embodiments, the backbone comprises the amino acid sequence of SEQ ID NO: 6 (QARILEADAEILRAYARILEAHAEILRAQ; Coil29 (PDB 3J89)). In some embodiments, the backbone has a coiled coil structure. In some embodiments, the backbone has a structure of a helical filament formed around a central axis. In some embodiments, the N-terminus of each backbone is positioned at the exterior of the helical filament. In some embodiments, each of the at least one glycomimetic peptide is capable of binding to a lectin. In some embodiments, the lectin comprises CD169. In some embodiments, the glycomimetic peptide is linked to the backbone by peptide linker. In some embodiments, the peptide linker comprises an amino acid sequence selected from SEQ ID NO: 9 (Gn wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 29 (KSGSG), SEQ ID NO: 30 (KKSGSG), and SEQ ID NO: 31 (EAAAK)₂. In some embodiments, the at least one glycomimetic peptide comprises a linear peptide, a branched peptide, or a combination thereof. In some embodiments, the branched peptide comprises at least 3 or 4 branches. In some embodiments, the branched peptide is linked to the backbone by peptide linker comprising at least one lysine. In some embodiments, the glycomimetic peptide comprises the amino acid sequence of SEQ ID NO: 25 [NPSHPLSGGGGS], or SEQ ID NO: 26 [(NPSHPLSGGGGS)₂K], or SEQ ID NO: 27 [((NPSHPLSGGGGS)₂K)₂], or a combination thereof. In some embodiments, the nanofiber comprises 2 to 10, 2 to 8, 2 to 6, or 2 to 4 glycomimetic peptides. In some embodiments, the antigen comprises a FP B-cell epitope. In some embodiments, the FP B-cell epitope comprises the amino acid sequence of SEQ ID NO: 28 (AVGIGAVFL). In some embodiments, the antigen comprises a B cell epitope in TNF, or IL-17, or phosphorylcholine, or a complement C3dg, or a B cell epitope in complement C5a, or a peptide comprising the amino acid sequence of SEQ ID NO: 45. In some embodiments, the nanofiber comprises 2 to 20, 2 to 15, 2 to 12, 2 to 10, 2 to 8, or 2 to 6 antigens. In some embodiments, the helical linker comprises a peptide comprising the amino acid sequence selected from SEQ ID NO: 9 (Gn wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), and SEQ ID NO: 31 (EAAAK)₂. In some embodiments, the nanofiber further comprises one or more capping peptides. In some embodiments, the capping peptide comprises an amino acid sequence selected from SEQ ID NOs: 32, 33, 34, 42, 43, 44, or a combination thereof. In some embodiments, the nanofiber is 50 nm to 600 nm in length.

In another aspect, provided herein is a method of immunizing a subject. The method may include administering to the subject a therapeutically effective amount of a composition as detailed herein. In some embodiments, the method further includes administering an adjuvant to the subject. In some embodiments, the method further includes administering a vaccine to the subject. In some embodiments, the vaccine is retained in the lymph nodes for at least 6 hours, at least 12 hours, at least 36 hours, at least 48 hours after administration. In some embodiments, the composition boosts germinal center reactions, thereby promoting a more robust immune response to the vaccine in the subject compared to a control. In some embodiments, the subject produces more IL-4 producing T cells after administration, compared to a control. In some embodiments, the subject produces more IFNγ producing T cells after administration, compared to a control. In some embodiments, the subject produces more antigen-binding antibodies or FP-binding antibodies after administration, compared to a control. In some embodiments, the subject is compared to the control at least 1 week, at least 2 weeks, at least 3 weeks, at least 4 weeks, at least 5 weeks, at least 6 weeks, or at least 7 weeks after administration of the nanofiber. In some embodiments, the vaccine comprises an HIV vaccine. In some embodiments, the at least one nanofiber is administered prior to the vaccine. In some embodiments, the at least one nanofiber is administered at least 1 week, at least 2 weeks, at least 3 weeks, at least 4 weeks, at least 5 weeks, at least 6 weeks, or at least 7 weeks, prior to the vaccine. In some embodiments, a composition as detailed herein primes the immune system of the subject prior to receiving the vaccine. In some embodiments, the at least one nanofiber is administered concurrently with the vaccine. In some embodiments, the at least one nanofiber is administered after the vaccine.

The disclosure provides for other aspects and embodiments that will be apparent in light of the following detailed description and accompanying figures.

### BRIEF DESCRIPTION OF THE DRAWINGS

FIG. 1. Nanostructure of Coiled-Coil Fusion Peptide Immunogens. Schematic of FP-Coil29 nanofibers based on the structure of Coil29 (PDB 3J89) incorporating FP peptides (orange) adjacent to helical linkers (turquoise) and glycomimetic peptide (yellow).

FIG. 2. Molecular Structure of Linear and Branched Glycomimetic Peptides. Glycomimetic peptides (yellow) displayed adjacent to the Coil29 sequence (blue) with either a linear peptide linker (Glyco-Coil29) or after branching lysine resides (Glyco4-Coil29).

FIG. 3. AFM images of Fusion Peptide-Bearing Nanofibers. Solutions of FP-Coil29 nanofibers were formed in PBS at 2 mM, diluted 10-fold in water, and deposited on mica substrates prior to imaging by AFM. Images displayed above are representative height maps.

FIG. 4. Viscometry of Fusion Peptide-Bearing Nanofibers. Viscosity of FP-Coil29 nanofibers formulated with capping and glycomimetic peptides was measured using a using a viscometry shear stress ramp from 0.1 to 1000 Hz.

FIG. 5. Drainage of Fluorescently Labeled Nanofibers to Lymph Nodes at 48 hours post-injection. Inguinal lymph nodes which drain directly from tail base subcutaneous injection sites were excised and imaged by IVIS to detect Alexa647-labeled peptides.

FIGS. 6A-6C. Quantification of Nanomaterial Accumulation in Lymph Nodes. Quantification of radiant efficiency in lymph nodes relative to naïve mice after ex vivo imaging by IVIS. Groups were compared by Ordinary One-Way ANOVA at each time point (6 hours in FIG. 6A, 12 hours in FIG. 6B, and 48 hours in FIG. 6C) with Dunnet's multiple comparison testing for pairwise comparisons. *p<0.05, ****p<0.0001.

FIG. 7. Scrambled Glycomimetic Peptide Viscometry. Viscosity of Capped FP-Coil29 nanofibers formulated scrambled and unscrambled glycomimetic peptides was measured using a using a viscometry shear stress ramp from 0.1 to 1000 Hz.

FIGS. 8A-8C. Glycomimetic Peptide Sequence Promotes Lymph Node Accumulation. Quantification of radiant efficiency in lymph nodes relative to naïve mice after ex vivo imaging by IVIS. Capped FP-Coil29+Glyco4-Coil29 is reproduced from FIG. 5 for comparison. Groups were compared by an unpaired t-test at each time point (6 hours in FIG. 8A, 12 hours in FIG. 8B, and 48 hours in FIG. 8C). ***p<0.001, ****p<0.0001.

FIG. 9. Glycomimetic Peptide Sequence Promotes Germinal Center Reactions. Mice were immunized via subcutaneous tail base injection 3 times in 2-week intervals and sacrificed 1 week after the final immunization. Draining inguinal lymph nodes were harvested and processed for flow cytometry to detect B220+CD19+GL7hi germinal center B cells. Groups were compared using a Mann Whitney non-parametric test. *p<0.05.

FIG. 10. ELISA Detection of FP-Reactive Antibodies via Multiple Coating Methods. FP-reactive antibodies from murine sera were detected via binding to 3 different peptide ELISA coatings utilizing N- and C-terminal biotinylated fusions, direct adsorption to ELISA plates, or via antibody captured SOSIP trimers.

FIG. 11. Antibody Responses to Unadjuvanted Fusion Peptide Immunogens. Mice were immunized via subcutaneous tail base injection 3 times in 2-week intervals and sacrificed 1 week after the final immunization. Groups were compared using a repeated measures one-way ANOVA. *p<0.05.

FIG. 12. T Cell Responses to Fusion Peptide Immunogens. Mice were immunized via subcutaneous tail base injection 3 times in 2-week intervals and sacrificed 1 week after the final immunization. Splenic lymphocytes were harvested and stimulated with the peptides shown below individual bars on the graph above. After 48 hours of incubation, IL-4 and IFNγ producing lymphocytes were detected by ELISPOT. Groups were compared by mixed-effects analysis and found to vary significantly by immunization and stimulating peptide (p=0.006). Responses to each stimulating peptide were then compared across groups using Dunnett's multiple comparison testing. **p<0.01.

FIG. 13. Antibody Responses to Fusion Peptide Induced by Heterologous Immunizations. Serum IgG binding to N-terminally displayed FP measured by ELISA from biweekly blood collected from mice immunized as shown by arrows above graph. Groups were compared by a non-parametric repeated measure's analysis (Friedman's test). *p<0.05, **p<0.01.

FIG. 14. Antibody Responses to SOSIP Trimers Induced by Heterologous Immunizations. SOSIP binding antibodies were measured after FP priming (week 5) and after multiple doses of SOSIP (week 11). Groups were compared using repeated measures one-way ANOVA and were not significantly different (p=0.34).

FIG. 15. Antibody Binding to C-terminally Displayed Fusion Peptide Induced by Heterologous Immunizations. Serum IgG binding to C-terminally displayed FP measured by ELISA from biweekly blood collected from mice immunized as shown by arrows above graph. Groups were compared by a non-parametric repeated measure's analysis (Friedman's test). **p<0.01, ****p<0.0001.

FIG. 16. Antibody Binding to Surface Bound Fusion Peptide Induced by Heterologous Immunizations. Serum IgG binding to directly adsorbed FP measured by ELISA from biweekly blood collected from mice immunized as shown by arrows above graph. Groups were compared by a non-parametric repeated measure's analysis (Friedman's test). *p<0.05.

FIG. 17. Antibody Binding to Disparate FP Conformations after Heterologous Boosting. Antibody response from N-terminal, C-terminal, and directly adsorbed FP ELISA were added to generate a quantification of total IgG. Groups were compared by a non-parametric repeated measure's analysis (Friedman's test). **p<0.01, ****p<0.0001.

FIG. 18. Antibody Binding Landscapes after Heterologous Immunization Regimens. Heat map representation of antibody titers to various coatings after immunization with FP immunogens. Groups were compared to FP-Coil29 using a non-parametric repeated measure's analysis (Friedman's test). Comparisons were made for each coating as indicated on individual rows and significance over course of the regimen is shown in the far-right column. *p<0.05, **p<0.01, ****p<0.0001.

FIGS. 19A-19B. Antibody Frequency and Binding Strength to Fusion Peptide Measured by SPR. Antibody binding to C-terminal FP was measured by Surface Plasmon Resonance. The relative magnitude of antibodies present in samples of equal IgG magnitude is displayed in FIG. 19A, and the dissociation rate of these antibodies is shown in FIG. 19B.

FIG. 20. Length of FP nanofibers. FP nanofibers imaged by AFM were measured using ImageJ and lengths were compared using One-way ANOVA. Capped and Uncapped formulations were compared using Bonferroni post-hoc testing.****p<0.0001.

FIG. 21. Dose Optimization of Glyco4-Coil29 Content in FP Nanofibers. FP-Coil29 immunizations were given at 0, 2, and 4 weeks and blood was sampled from mice at 7 weeks after the first immunization. Antibody binding to FP and Glyco peptides was measured by ELISA. Antibody responses to each immunization were compared using 2-way ANOVA and response to FP and Glyco were compared across groups. *p<0.05, **p<0.01, ***p<0.001.

### DETAILED DESCRIPTION

Detailed herein are immunogenic compositions that may be used as a vaccine or in combination with a vaccine. As detailed herein, highly multivalent immunogens activate low-affinity B cells, which alters the binding breadth of elicited antibodies. Nanofibers bearing HIV fusion peptides were designed, and their capacity to produce high magnitude antibodies to FP that can bind FP in a variety of conformations was tested. The nanofibers detailed herein include a backbone that comprises a fibril of self-assembling peptides. At least one glycomimetic peptide is linked to the backbone. Additionally or alternatively, at least one antigen such as a FP B-cell epitope is linked to the backbone. Further provided herein are methods of immunizing a subject by administering the nanofibers to the subject. The nanofibers may boost germinal center reactions and promote a more robust immune response.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “adjuvant” refers to a compound or mixture that enhances the immune response to an antigen. Adjuvants may contain a substance to protect the antigen from rapid catabolism, such as aluminum hydroxide or a mineral oil, and also a protein derived from lipid A, Bortadella pertussis, or Mycobacterium tuberculosis. Suitable adjuvants may be commercially available and include, for example, complete or incomplete Freund's adjuvant; AS-2; aluminum salts such as aluminum hydroxide (as a gel, where appropriate) or aluminum phosphate; calcium salts, iron salts, or zinc salts; an insoluble suspension of acylated tyrosine; acylated sugars; cationically or anionically derivatized polysaccharides; polyphosphazenes; biologically degradable microspheres; monophosphoryl lipid A, cytokines such as GM-CSF, Interleukin-2, Interleukin-7, and Interleukin-12.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject, or a sample therefrom, whose disease state is known. The subject, or sample therefrom, may be healthy, diseased, diseased prior to treatment, diseased during treatment, or diseased after treatment, or a combination thereof.

“Immunogenicity” refers to the ability of an antigen to induce an immune response and includes the intrinsic ability of an antigen to generate antibodies in a subject.

“Polynucleotide” as used herein can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. Secondary structure may include beta-sheet and alpha-helices. These structures are commonly known as domains, for example, enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. In some embodiments, a carrier includes a solution at neutral pH. In some embodiments, a carrier includes a salt. In some embodiments, a carrier includes a buffered solution. In some embodiments, a carrier includes phosphate buffered saline solution.

“Sample” or “test sample” as used herein can mean any sample in which the presence and/or level of a target is to be detected or determined or a portion from a subject or portion of an immunogenic composition as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” as used herein can mean a mammal that wants or is in need of the herein described immunogenic compositions. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant.

“Treatment” or “treating,” when referring to protection of a subject from a disease, means preventing, suppressing, repressing, ameliorating, or completely eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (for example, hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of +2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

### 2. Immunogenic Compositions

Described herein is a platform for vaccination or treatment based on alpha-helical peptides assembled into nanofibers. The nanofibers include a backbone. At least one glycomimetic peptide and/or at least one epitope is attached to the backbone. The backbone comprises a peptide fibril, comprised of self-assembling peptides, that folds into a complex alpha-helix-based nanofiber where individual peptide coils run perpendicular to the axis of a long fibril. The resultant nanostructure is composed of thousands of individual peptides or more. Nanofibers have been observed to be up to several microns long. The self-assembling peptide may be extended N-terminally with a flexible spacer and an immune epitope. The self-assembling peptide may be linked to a glycomimetic peptide.

In some embodiments, the composition does not further comprise an adjuvant. In some embodiments, the composition further comprises an adjuvant. In some embodiments, the nanofiber is an adjuvant.

Multiple epitope-bearing and/or glycomimetic peptide-bearing self-assembling peptides are then co-assembled into nanofibers composed not of B-sheets, but of a-helices. Coiled coil folding requires more extensive design considerations compared to β-sheet fibrillization, as both inter-helical interactions as well as those between the C-terminus and the main chain must be considered. This folding strategy allows for greater structural control and tunable rates of assembly and disassembly. This control may be useful in optimizing the materials' trafficking and engagement of specific immune cells in vivo.

a. Nanofibers

In some embodiments, the nanofiber comprises a backbone, at least one glycomimetic peptide linked to the backbone, and at least one epitope linked to the backbone. In some embodiments, the nanofiber comprises a backbone, and at least one glycomimetic peptide linked to the backbone. In some embodiments, the nanofiber comprises a backbone, and at least one epitope linked to the backbone.

In some embodiments, the composition includes a first nanofiber comprising a backbone, at least one glycomimetic peptide linked to the backbone, and at least one epitope linked to the backbone; and a second nanofiber comprising a backbone and at least one glycomimetic peptide linked to the backbone. In some embodiments, the composition includes a first nanofiber comprising a backbone, at least one glycomimetic peptide linked to the backbone, and at least one epitope linked to the backbone; and a second nanofiber comprising a backbone and at least one epitope linked to the backbone. In some embodiments, the composition includes a first nanofiber comprising a backbone and at least one glycomimetic peptide linked to the backbone, and a second nanofiber comprising a backbone and at least one epitope linked to the backbone. In some embodiments, the composition includes a first nanofiber comprising a backbone, at least one glycomimetic peptide linked to the backbone, and at least one epitope linked to the backbone; a second nanofiber comprising a backbone and at least one glycomimetic peptide linked to the backbone; and a third nanofiber comprising a backbone and at least one epitope linked to the backbone. The nanofibers may each be about 50 nm to 600 nm in length.

### i) Backbone

The backbone of the nanofiber comprises a peptide fibril. Peptide fibrils are described in, for example, International Patent Application No. PCT/US2017/025596 filed Mar. 31, 2017, and published Oct. 5, 2017 as WO/2017/173398, and US2020/0390882 published Dec. 17, 2020, each of which is incorporated by reference herein. The peptide fibril comprises a plurality of self-assembling peptides.

The peptide fibril backbone can have a length of at least, at most, or exactly 0.01, 0.05, 0.1, 0.15, 0.20, 0.25, 0.5, 1, 5, 10, 20, 30, 40, 50, 75, 100, 125, 150, 175, 200, 225, 250, or 300 μm, including all values and ranges there between. In some embodiments, the peptide fibril backbone is at least 100, 150, 200, 250, 300, or 350 nanometers in length. In some embodiments, the peptide fibril backbone is less than 10, 5, or 2 μm in length. In certain aspects, the peptide fibril backbone has a molecular weight of at least 100, 500, 1,000, 5,000, 10,000, 100,000 Da to 1×10⁶, 1×10⁷, 7×10⁸ Da, including all values and ranges there between. The peptide fibril backbone can have a diameter or width of at least, at most, or exactly 5, 10, 15, 20, 25, or 30 nm. In some embodiments, the peptide fibril backbone is from 5 to 30 nm, or from 10 to 30 nm in diameter or width.

The peptide fibril comprises a plurality of self-assembling peptides. As used herein, the term “self-assembling peptide” refers to peptides that are able to spontaneously associate and form stable structures.

The self-assembling peptide may comprise an amino acid sequence of bXXXb (SEQ ID NO: 1), wherein X is independently any amino acid, and b is independently any positively charged amino acid. In some embodiments, b is independently selected from Arg and Lys. In some embodiments, b is Arg. In some embodiments, bXXXb (SEQ ID NO: 1) is RAYAR (SEQ ID NO: 2). In some embodiments, bXXXb (SEQ ID NO: 1) is KAYAK (SEQ ID NO: 3). In some embodiments, the self-assembling peptide comprises the sequence of RXXXR (SEQ ID NO: 4), wherein X is any amino acid. The self-assembling peptide may comprise an amino acid sequence of ZnbXXXbZm (SEQ ID NO: 5), wherein b is independently any positively charged amino acid, Z is independently any amino acid, X is independently any amino acid, n is an integer from 0 to 20 or 1 to 20, and m is an integer from 0 to 20 or 1 to 20. In some embodiments, n is an integer from 5 to 15, and m is an integer from 5 to 15. In some embodiments, the self-assembling peptide comprises a glutamine at the C-terminus. In some embodiments, the self-assembling peptide comprises a glutamine at the N-terminus. The self-assembling peptide may include at least, at most, or exactly 5, 10, 15, 16, 17, 18, 19, 20, 21, 22, 23, 24, 25, 26, 27, 28, 29, 30, 31, 32, 33, 34, 35, or 40 amino acids. In some embodiments, the self-assembling peptide comprises from 5 to 40 amino acids in length.

In some embodiments, the self-assembling peptide comprises an amino acid sequence of QARILEADAEILRAYARILEAHAEILRAQ (SEQ ID NO: 6) or QAKILEADAEILKAYAKILEAHAEILKAQ (SEQ ID NO: 7) or ADAEILRAYARILEAHAEILRAQ (SEQ ID NO: 8) or a polypeptide with at least 75%, at least 80%, at least 85%, at least 90%, or at least 95% identity thereto. In some embodiments, the self-assembling peptide comprises an amino acid sequence of QARILEADAEILRAYARILEAHAEILRAQ (SEQ ID NO: 6) or QAKILEADAEILKAYAKILEAHAEILKAQ (SEQ ID NO: 7) or ADAEILRAYARILEAHAEILRAQ (SEQ ID NO: 8) or a variant thereof. In some embodiments, the self-assembling peptide comprises an amino acid sequence of QARILEADAEILRAYARILEAHAEILRAQ (SEQ ID NO: 6). In some embodiments, the self-assembling peptide comprises an amino acid sequence of QAKILEADAEILKAYAKILEAHAEILKAQ (SEQ ID NO: 7). In some embodiments, the self-assembling peptide comprises an amino acid sequence of ADAEILRAYARILEAHAEILRAQ (SEQ ID NO: 8).

In some embodiments, the backbone comprises an amino acid sequence selected from QARILEADAEILRAYARILEAHAEILRAQ (SEQ ID NO: 6; Coil29; PDB 3J89), or QAKILEADAEILKAYAKILEAHAEILKAQ (SEQ ID NO: 7), ADAEILRAYARILEAHAEILRAQ (SEQ ID NO: 8), or a polypeptide with at least 75%, at least 80%, at least 85%, at least 90%, or at least 95% identity thereto, or a combination thereof. In some embodiments, the backbone comprises a plurality of peptides having an amino acid sequence selected from QARILEADAEILRAYARILEAHAEILRAQ (SEQ ID NO: 6; Coil29; PDB 3J89), QAKILEADAEILKAYAKILEAHAEILKAQ (SEQ ID NO: 7), ADAEILRAYARILEAHAEILRAQ (SEQ ID NO: 8), or a polypeptide with at least 75%, at least 80%, at least 85%, at least 90%, or at least 95% identity thereto, or a combination thereof. In some embodiments, the backbone comprises a peptide having the amino acid sequence of QARILEADAEILRAYARILEAHAEILRAQ (SEQ ID NO: 6; Coil29; PDB 3J89). In some embodiments, the backbone comprises a plurality of peptides having the amino acid sequence of QARILEADAEILRAYARILEAHAEILRAQ (SEQ ID NO: 6; Coil29; PDB 3J89).

Self-assembling peptides may further comprise other compounds, for example, immunogenic peptides.

In some embodiments, the self-assembling polypeptide includes a modification to the C-terminus, to the N-terminus, or to both the C-terminus and N-terminus. N-terminal modifications may include, for example biotin and actyl. C-terminal modifications may include, for example, amide.

Each self-assembling peptide may include an acetylated N-terminus (Ac), or an amidated C-terminus (NH2), or a combination thereof. In some embodiments, the self-assembling peptide comprises an amino acid sequence selected from SEQ ID NOs: 35, 36, 37, or a polypeptide with at least 75%, at least 80%, at least 85%, at least 90%, or at least 95% identity thereto. In some embodiments, the self-assembling peptide comprises an amino acid sequence selected from SEQ ID NOs: 35, 36, 37, or a variant thereof. In some embodiments, the self-assembling peptide comprises the amino acid sequence of SEQ ID NO: 35. In some embodiments, the self-assembling peptide comprises an amino acid sequence of SEQ ID NO: 36. In some embodiments, the self-assembling peptide comprises an amino acid sequence of SEQ ID NO: 37.

In some embodiments, the backbone comprises an amino acid sequence selected from SEQ ID NOs: 35, 36, 37, or a polypeptide with at least 75%, at least 80%, at least 85%, at least 90%, or at least 95% identity thereto, or a combination thereof. In some embodiments, the backbone comprises a plurality of peptides having an amino acid sequence selected from SEQ ID NOs: 35, 36, 37, or a polypeptide with at least 75%, at least 80%, at least 85%, at least 90%, or at least 95% identity thereto, or a combination thereof. In some embodiments, the backbone comprises a peptide having the amino acid sequence of Ac-QARILEADAEILRAYARILEAHAEILRAQ-NH2 (SEQ ID NO: 35). In some embodiments, the backbone comprises a plurality of peptides having the amino acid sequence of Ac-QARILEADAEILRAYARILEAHAEILRAQ-NH2 (SEQ ID NO: 35).

The peptides described herein can be chemically synthesized using standard chemical synthesis techniques. In some embodiments the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides described herein. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85:2149-2156, and Stewart et al. (1984) Solid Phase Peptide Synthesis, 2nd ed. Pierce Chem. Co., Rockford, Ill. In some embodiments, the self-assembling peptide is synthesized by a solid phase peptide synthesis.

Each self-assembling peptide comprises or forms an alpha helix. The plurality of self-assembling peptides may form a peptide fibril in the form of a helical filament. The helical filament may be formed around a central axis or core. The plurality of self-assembling peptides may form a peptide fibril in the form of a coiled coil. In some embodiments, the N-terminus of each self-assembling peptide is positioned at the exterior of the helical filament. An example of the self-assembling peptides formed into a peptide fibril is also disclosed in Egelman et al. Structure 2015, 23, 280-289, incorporated herein by reference. In some embodiments, the backbone has a coiled coil structure. In some embodiments, the backbone has a structure of a helical filament formed around a central axis. In some embodiments, the N-terminus of each backbone is positioned at the exterior of the helical filament. For example, a self-assembling peptide such as Coil29 may form an alpha helix, having two hydrophobic stripes running down the axis of the helix. This may allow it to stack on top of itself.

### ii) Glycomimetic Peptide

In some embodiments, at least one glycomimetic peptide is linked to the backbone. The glycomimetic peptide may be capable of binding to a lectin. In some embodiments, the lectin comprises CD169. The glycomimetic peptide may comprise a linear peptide, a branched peptide, or a combination thereof. The branched peptide may include at least 2, at least 3, at least 4, or at least 5 branches.

The glycomimetic peptide may comprise the amino acid sequence of SEQ ID NO: 25 [NPSHPLSGGGGS], or SEQ ID NO: 26 [(NPSHPLSGGGGS)₂K], or SEQ ID NO: 27 [((NPSHPLSGGGGS)₂K)₂], or a combination thereof. The glycomimetic peptide may be directly or indirectly conjugated to a self-assembling peptide of the backbone. The nanofiber may comprise a plurality of glycomimetic peptides coupled thereto. In some embodiments, the nanofiber comprises 2 to 10, 2 to 8, 2 to 6, or 2 to 4 glycomimetic peptides. In some embodiments, the backbone is coupled to a plurality of glycomimetic peptides. A self-assembling peptide of the backbone may be conjugated to a glycomimetic peptide.

iii) Antigens

In some embodiments, at least one antigen is linked to the backbone. The antigen may be directly or indirectly conjugated to a self-assembling peptide of the backbone. The nanofiber may comprise a plurality of antigens coupled thereto. In some embodiments, the nanofiber comprises 2 to 20, 2 to 15, 2 to 12, 2 to 10, 2 to 8, or 2 to 6 antigens. In some embodiments, the backbone is coupled to a plurality of antigens. A self-assembling peptide of the backbone may be conjugated to an antigen. In some embodiments, each self-assembling peptide is conjugated to an antigen.

The antigen may be conjugated or coupled to a self-assembling peptide of the backbone by any means known in the art, including, for example, click chemistry, Spytag/Spycatcher, oxime ligation, condensation reactions. In some embodiments, the antigen is covalently coupled to the self-assembling peptide. In some embodiments, the antigen is attached to the self-assembling peptide through a thiol reactive group. The antigen may be covalently coupled to a terminus of the self-assembling peptide. In some embodiments, the antigen is covalently coupled to the N-terminus of the self-assembling peptide. The conjugation of the antigen to the N-terminus of the self-assembling peptide may orient the antigen towards the exterior of the helical peptide fibril. In some embodiments, the antigens are exposed on the exterior surface of the peptide fibril. In some embodiments, the antigens are exposed on the exterior surface of the helical filament of the peptide fibril. In some embodiments, the antigen is covalently coupled to the self-assembling peptide. In some embodiments, the antigen is covalently coupled to a terminus of the self-assembling peptide. In some embodiments, the antigens are covalently coupled to the amino terminus of the self-assembling peptide. In some embodiments, the antigens are covalently coupled to the carboxy terminus of the self-assembling peptide.

In some embodiments, the nanofiber comprises the same antigen. In some embodiments, the nanofiber comprises at least two different antigens. The nanofiber may comprise at least, at most, or exactly 1, 2, 3, 4, 5, 6, 7, 8, 9, 10, 11, 12, 13, 14, 15, 16, 17, 18, 19, 20, 30, 40, 50, 100, 500, 1000, or 10,000 different antigens (or any derivable range therein). In some embodiments, the nanofiber includes n different antigens, wherein n is an integer from 1 to 10,000. The relative ratio of one antigen to another in the nanofiber may be at least, at most, or exactly 1, 2, 3, 4, 5, 6, 7, 8, 9, 10, 11, 12, 13, 14, 15, 16, 17, 18, 19, 20, 50, 100, 200, 300, 400, or 500 to 1, 2, 3, 4, 5, 6, 7, 8, 9, 10, 11, 12, 13, 14, 15, 16, 17, 18, 19, 20, 50, 100, 200, 300, 400, or 500 (or any derivable range therein).

In some embodiments, the antigens are exposed on the surface of the nanofiber. In certain aspects the ratio of antigen to self-assembling peptide is 1:1000, 1:100:1:10, or 1:1, including all values and ranges there between.

As used herein, the term “antigen” is a molecule capable of being bound by an antibody or T-cell receptor. The term “antigen”, as used herein, also encompasses T-cell epitopes. An antigen also refers to a molecule against which a subject can initiate a humoral and/or cellular immune response leading to the activation of B-lymphocytes and/or T-lymphocytes. An antigen is capable of inducing a humoral immune response and/or cellular immune response leading to the production of B- and/or T-lymphocytes. The structural aspect of an antigen that gives rise to a biological response is referred to herein as an “antigenic determinant.” B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors. An antigenic determinant need not be a contiguous sequence or segment of protein and may include various sequences that are not immediately adjacent to one another. In some embodiments, the antigen contains or is linked to a Th cell epitope. An antigen can have one or more epitopes (B-epitopes and T-epitopes). Antigens may also be mixtures of several individual antigens.

Antigens can be any type of biologic molecule including, for example, simple intermediary metabolites, sugars, lipids, and hormones as well as macromolecules such as complex carbohydrates, phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, and other miscellaneous antigens. Antigens can be microbial antigens, such as viral, fungal, or bacterial; or therapeutic antigens such as antigens associated with cancerous cells or growths, or autoimmune disorders. In some embodiments, the antigen is selected from a small molecule, nucleotide, polynucleotide, peptide, polypeptide, protein, lipid, carbohydrate, other immunogenic molecules, and a combination thereof. In some embodiments, the plurality of antigens comprises a B cell epitope or T cell epitope. In some embodiments, the plurality of antigens comprises a B cell epitope and a T cell epitope. In some embodiments, the antigen comprises an autologous target. In some embodiments, the antigen comprises a cytokine. In certain compositions and methods, the antigen comprises a peptide. In some embodiments, the antigen comprises a peptide from 5 to 20 amino acids in length. The peptide may be at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80 90 or 100 amino acids (or any derivable range therein). In some embodiments, the peptide is from 5 to 20 amino acids in length. In some embodiments, the nanofiber is non-toxic.

### Viral Antigens.

Examples of viral antigens include, but are not limited to, retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, for example, hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, 80% E, and other Japanese encephalitis viral antigen components; rabies viral antigens such as rabies glycoprotein, rabies nucleoprotein and other rabies viral antigen components. See Fundamental Virology, Second Edition, e's. Fields, B. N. and Knipe, D. M. (Raven Press, New York, 1991) for additional examples of viral antigens.

In some embodiments, the antigen comprises an HIV antigen. For example, the antigen may comprise the fusion peptide (FP) of HIV-1 trimers, which is also referred to herein as the FP B-cell epitope. The FP B-cell epitope may comprise the amino acid sequence of SEQ ID NO: 28 (AVGIGAVFL). The nanofiber may comprise, for example, from 2 to 20, from 2 to 15, 2 to 12, from 2 to 10, from 2 to 8, or from 2 to 6 FP B-cell epitopes.

### Bacterial Antigens.

Bacterial antigens which can be used in the compositions and methods include, but are not limited to, gonorrhea bacterial antigens; pertussis bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; hemophilus influenza bacterial antigens such as capsular polysaccharides and other hemophilus influenza bacterial antigen components; anthrax bacterial antigens such as anthrax protective antigen and other anthrax bacterial antigen components; rickettsiae bacterial antigens such as romps and other rickettsiae bacterial antigen component. Also included with the bacterial antigens described herein are any other bacterial, mycobacterial, mycoplasmal, rickettsial, or chlamydial antigens. Bacterial antigens may include an antigen from a bacteria related to a bacterial infection, which are further detailed below.

In some embodiments, the antigen comprises a gonorrhea antigen. For example, the antigen may comprise a 2C7 epitope, which is from the LOS mimitope. The 2C7 epitope may comprise the amino acid sequence of IPVLDENGLFAP (SEQ ID NO: 45).

### Fungal Antigens.

Fungal antigens which can be used in the compositions and methods include, but are not limited to, Candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

### Parasite Antigens.

Examples of protozoa and other parasitic antigens include, but are not limited to, Plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; Leishmania major and other leishmaniae antigens such as gp63, lipophosphoglycan and its associated protein and other leishmanial antigen components; and Trypanosoma cruzi antigens such as the 75-77 kDa antigen, the 56 kDa antigen and other trypanosomal antigen components.

### Tumor Antigens.

Tumor antigens which can be used in the compositions and methods include, but are not limited to, telomerase components; multidrug resistance proteins such as P-glycoprotein; MAGE-1, alpha fetoprotein, carcinoembryonic antigen, mutant p53, immunoglobulins of B-cell derived malignancies, fusion polypeptides expressed from genes that have been juxtaposed by chromosomal translocations, human chorionic gonadotrpin, calcitonin, tyrosinase, papillomavirus antigens, gangliosides or other carbohydrate-containing components of melanoma or other tumor cells. It is contemplated that antigens from any type of tumor cell can be used in the compositions and methods described herein.

### Antigens Relating to Autoimmunity.

Antigens involved in autoimmune diseases, allergy, and graft rejection can be used in the compositions and methods. For example, an antigen involved in any one or more of the following autoimmune diseases or disorders can be used: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. An antigen can also be an altered peptide ligand useful in treating an autoimmune disease.

Examples of miscellaneous antigens which can be used in the compositions and methods include endogenous hormones such as luteinizing hormone, follicular stimulating hormone, testosterone, growth hormone, prolactin, and other hormones, drugs of addiction such as cocaine and heroin, and idiotypic fragments of antigen receptors such as Fab-containing portions of an anti-leptin receptor antibody.

In some embodiments, the antigen comprises an inflammatory bowel disease antigen. For example, the antigen may comprise an inflammatory bowel disease epitope selected from phosphorylcholine, complement C3dg, and/or B cell epitopes in complement C5a.

In some embodiments, the antigen comprises a Rheumatoid Arthritis antigen. For example, the antigen may comprise a Rheumatoid Arthritis epitope selected from B cell epitopes in TNF, and/or IL-17.

### iv) Linker

The nanofiber may further comprise at least one linker. The linker may be between the antigen and the backbone. The linker may be between the glycomimetic peptide and the backbone. In some embodiments, a linker is covalently attached to the backbone between the antigen and the self-assembling peptide. In some embodiments, a linker is covalently attached to the backbone between the glycomimetic peptide and the self-assembling peptide.

In some embodiments, the nanofiber includes more than one linker. In such embodiments, the linkers may be the same or different from one another. The nanofiber may include at least 1, at least 2, at least 3, at least 4, at least 5, at least 6, at least 7, at least 8, at least 9, or at least 10 linkers. The nanofiber may include less than 25, less than 20, less than 15, less than 10, or less than 5 linkers. The conjugate may include between 1 and 25, between 1 and 20, between 5 and 15, or between 1 and 5 linkers. The conjugate may include from 1 to 25, from 1 to 20, from 5 to 15, or from 1 to 5 linkers. The linker may be positioned at the C-terminus of the self-assembling peptide, at the N-terminus of the self-assembling peptide, or at both the N- and C-termini of the self-assembling peptide. In some embodiments, the linker is positioned at the N-terminus of the self-assembling peptide. Multiple linkers may be positioned adjacent to one another.

In some embodiments, the linker comprises glycine and serine. In some embodiments, the antigen and/or glycomimetic peptide is attached to the self-assembling peptide through a thiol reactive group in the linker. In embodiments including a glycomimetic peptide comprising a branched peptide, the branched peptide is linked to the backbone by peptide linker comprising at least one lysine. The antigen may be linked to the backbone by a helical linker. In some embodiments, the linker comprises oligoethylene glycol, polyethylene glycol, or an amino acid sequence selected from SEQ ID NO: 9 (Gn wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 29 (KSGSG), SEQ ID NO: 30 (KKSGSG), and SEQ ID NO: 31 (EAAAK)₂ or a combination thereof.

### v) Capping Peptide

In some embodiments, the nanofiber further includes at least one capping peptide. The capping peptides may be at the termini of the peptide nanofibers. Each peptide strand runs perpendicular to the long axis of the fiber, and in some embodiments, the capping peptide is at one or at both ends of the nanofiber. The capping peptide is combined with the self-assembling peptides during self-assembly, and it co-assembles with the other peptides in the nanofiber. The capping peptide may comprise the amino acid sequence of SEQ ID NO: 42, or SEQ ID NO: 43, or SEQ ID NO: 44, or a polypeptide with at least 75%, at least 80%, at least 85%, at least 90%, or at least 95% identity thereto, or a combination thereof. The capping peptide may include an acetylated N-terminus, or an amidated C-terminus, or a combination thereof. The capping peptide may comprise the amino acid sequence of SEQ ID NO: 32 (Ac-QSRITEADSEITRAYSRITEAHSEITRAQ-NH2) or SEQ ID NO: 33 (Ac-QARILEADAEILRAYAEILEAHAEILRAQ-NH2) or SEQ ID NO: 34 (Ac-QARTLESDAETLRSYARTLESHAETLRSQ-NH2), or a polypeptide with at least 75%, at least 80%, at least 85%, at least 90%, or at least 95% identity thereto, or a combination thereof. The nanofiber may include one or two capping peptides selected from SEQ ID NOs: 32, 33, 34, 42, 43, or 44, or a polypeptide with at least 75%, at least 80%, at least 85%, at least 90%, or at least 95% identity thereto. The capping peptides may form an alpha helix with each alpha helix having only one hydrophobic stripe, such that when they assemble onto the nanofiber, they terminate further fiber growth.

b. Immune Response and Immunoassays

As discussed above, the compositions and methods provided herein include evoking or inducing an immune response in a subject against an antigen. In one embodiment, the immune response can protect against or treat a subject having, suspected of having, or at risk of developing an infection or related disease, or a pathological condition such as cancer or autoimmunity. One use of the immunogenic compositions is to provide effective vaccines, such as cancer vaccines. The compositions detailed herein may induce an immune response. The immune response may be an antigen-specific immune response. In some embodiments, the antigen-specific immune response is temporary or not life-long. In some embodiments, the immune response comprises IgG1 antibody isotypes. In some embodiments, the immune response is an anti-cancer immune response. The immunogenic composition may have increased immunogenicity relative to a control. In some embodiments, the control comprises the antigen without a self-assembling peptide.

Further provided herein is the implementation of serological assays to evaluate whether and to what extent an immune response is induced or evoked by compositions. There are many types of immunoassays that can be implemented. Immunoassays include, but are not limited to, those described in U.S. Pat. No. 4,367,110 (double monoclonal antibody sandwich assay) and U.S. Pat. No. 4,452,901 (western blot), which are incorporated herein by reference. Other assays include immunoprecipitation of labeled ligands and immunocytochemistry, both in vitro and in vivo.

Immunoassays generally are binding assays. Certain immunoassays are the various types of enzyme linked immunosorbent assays (ELISAs) and radioimmunoassays (RIA) known in the art. Immunohistochemical detection using tissue sections is also useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

c. Protective Immunity

In some embodiments, proteinaceous compositions confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein in the specification and in the claims section that follows, the term polypeptide or peptide refer to a stretch of amino acids covalently linked there amongst via peptide bonds. Different polypeptides may have different functionalities. While according to one aspect, a polypeptide is derived from an immunogen designed to induce an active immune response in a recipient, according to another aspect, a polypeptide is derived from an antibody which results following the elicitation of an active immune response in, for example, an animal, and which can serve to induce a passive immune response in the recipient. In both cases, however, the polypeptide is encoded by a polynucleotide according to any possible codon usage.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, carbohydrate, or polypeptide in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an antigen.

As used herein “passive immunity” refers to any immunity conferred upon a subject without administration of an antigen to the subject. “Passive immunity” therefore includes, but is not limited to, administration of activated immune effectors including cellular mediators or protein mediators (for example, monoclonal and/or polyclonal antibodies) of an immune response. A monoclonal or polyclonal antibody composition may be used in passive immunization for the prevention or treatment of infection by organisms that carry the antigen recognized by the antibody. An antibody composition may include antibodies that bind to a variety of antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as gram-positive bacteria, gram-negative bacteria, including but not limited to staphylococcus bacteria.

Passive immunity may be imparted to a patient or subject by administering to the patient immunoglobulins (Ig) and/or other immune factors obtained from a donor or other non-patient source having a known immunoreactivity. In other aspects, an antigenic composition as detailed herein can be administered to a subject who then acts as a source or donor for globulin, produced in response to challenge with the antigenic composition (“hyperimmune globulin”), that contains antibodies directed against Staphylococcus or other organism. A subject thus treated would donate plasma from which hyperimmune globulin would then be obtained, via conventional plasma-fractionation methodology, and administered to another subject in order to impart resistance against or to treat staphylococcus infection. Hyperimmune globulins are particularly useful for immune-compromised individuals, for individuals undergoing invasive procedures or where time does not permit the individual to produce their own antibodies in response to vaccination. See U.S. Pat. Nos. 6,936,258, 6,770,278, 6,756,361, 5,548,066, 5,512,282, 4,338,298, and 4,748,018, each of which is incorporated herein by reference in its entirety, for exemplary methods and compositions related to passive immunity.

For purposes of this specification and the accompanying claims the terms “epitope” and “antigenic determinant” are used interchangeably to refer to a site on an antigen to which B and/or T cells respond or recognize. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by 5H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

As used herein and in the claims, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins.

Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains.

In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies can be produced by hyperimmunization of an appropriate donor with the antigen or ex-vivo by use of primary cultures of splenic cells or cell lines derived from spleen (Anavi, 1998; Huston et al., 1991; Johnson et al., 1991; Mernaugh et al., 1995).

As used herein and in the claims, the phrase “an immunological portion of an antibody” includes a Fab fragment of an antibody, a Fv fragment of an antibody, a heavy chain of an antibody, a light chain of an antibody, a heterodimer consisting of a heavy chain and a light chain of an antibody, a variable fragment of a light chain of an antibody, a variable fragment of a heavy chain of an antibody, and a single chain variant of an antibody, which is also known as scFv. In addition, the term includes chimeric immunoglobulins which are the expression products of fused genes derived from different species, one of the species can be a human, in which case a chimeric immunoglobulin is said to be humanized. Typically, an immunological portion of an antibody competes with the intact antibody from which it was derived for specific binding to an antigen.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

d. Pharmaceutical Compositions

Further provided herein are methods for immunization against microbial infections or viral infections, which in some embodiments may be used in, for example, for the treatment of cancer. As such, contemplated are vaccines and therapeutics for use in active immunization of subjects.

The preparation of vaccines that contain polypeptide or peptide sequence(s) as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, vaccines are formulated with a combination of substances, as described in U.S. Pat. Nos. 6,793,923 and 6,733,754, which are incorporated herein by reference.

Vaccines and therapeutics may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The compositions described herein may be formulated into a pharmaceutical composition as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Typically, compositions are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection and the like. The dosage of the vaccine will depend on the route of administration and will vary according to the size and health of the subject.

The compositions and related methods, particularly administration of a peptide fibril/antigen complex may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline or various combinations of antibiotics.

With respect to cancer treatments, the current methods and compositions described herein may be used in combination with traditional cancer therapies such as surgery, chemotherapeutics, and/or radiation therapy. Cancer therapies also include a variety of combination therapies with both chemical and radiation-based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

In yet another embodiment, the treatment is a gene therapy. In certain embodiments, the therapeutic gene is a tumor suppressor gene. A tumor suppressor gene is a gene that, when present in a cell, reduces the tumorigenicity, malignancy, or hyperproliferative phenotype of the cell. This definition includes both the full-length nucleic acid sequence of the tumor suppressor gene, as well as non-full length sequences of any length derived from the full length sequences. It being further understood that the sequence includes the degenerate codons of the native sequence or sequences which may be introduced to provide codon preference in a specific host cell. Examples of tumor suppressor nucleic acids within this definition include, but are not limited to APC, CYLD, HIN-I, KRAS2b, pló, pl9, p21, p27, p27mt, p53, p57, p73, PTEN, Rb, Uteroglobin, Skp2, BRCA-I, BRCA-2, CHK2, CDKN2A, DCC, DPC4, MADR2/JV18, MENI, MEN2, MTSI, NFI, NF2, VHL, WRN, WTI, CFTR, C-CAM, CTS-I, zacl, scFV, MMACI, FCC, MCC, Gene 26 (CACNA2D2), PL6, Beta* (BLU), Luca-1 (HYALI), Luca-2 (HYAL2), 123F2 (RASSFI), 101F6, Gene 21 (NPRL2), or a gene encoding a SEM A3 polypeptide and FUSI. Other exemplary tumor suppressor genes are described in a database of tumor suppressor genes at www.cise.ufl.edu/˜yyl/HTML-TSGDB/Homepage.litml. This database is herein specifically incorporated by reference into this and all other sections of the present application. Nucleic acids encoding tumor suppressor genes, as discussed above, include tumor suppressor genes, or nucleic acids derived therefrom (for example, cDNAs, CRNAs, mRNAs, and subsequences thereof encoding active fragments of the respective tumor suppressor amino acid sequences), as well as vectors comprising these sequences. One of ordinary skill in the art would be familiar with tumor suppressor genes that can be applied.

In one aspect, it is contemplated that a nanofiber therapy as detailed herein is used in conjunction with an additional treatment. Alternatively, the therapy may precede or follow the other agent treatment by intervals ranging from minutes to weeks. In embodiments where the other agents and/or a proteins is administered separately, one would generally ensure that a significant period of time did not expire between the time of each delivery, such that the agent and antigenic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6, or 7) to several weeks (1, 2, 3, 4, 5, 6, 7, or 8) lapse between the respective administrations.

Various combinations may be employed, for example antibiotic or vaccine therapy is “A” and the immunogenic composition detailed herein is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the immunogenic compositions to a patient/subject will follow general protocols for the administration of such compounds, taking into account the toxicity, if any. It is expected that the treatment cycles would be repeated as necessary. It also is contemplated that various standard therapies, such as hydration, may be applied in combination with the described therapy.

In some embodiments, pharmaceutical compositions are administered to a subject. Different aspects involve administering an effective amount of a composition to a subject. In some embodiments, immunogenic compositions may be administered to the patient to protect against infection by one or more microbial or viral pathogens. Additionally, such compounds can be administered in combination with an antibiotic or other known anti-microbial therapy or antiviral therapy. Such compositions will generally be dissolved or dispersed in a pharmaceutically acceptable carrier or aqueous medium. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-cancer agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including creams, lotions, mouthwashes, inhalants and the like.

The active compounds can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Administration of the compositions will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. In some embodiments, the immunogenic composition is administered to the subject intravenously, intraarterially, intraperitoneally, subcutaneously, intranasally, intramuscularly, or intratumorally.

For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

e. Methods

### i) Methods of Immunizing a Subject

Further provided herein are methods of immunizing a subject. The method may include administering to the subject a therapeutically effective amount of the immunogenic composition detailed herein. In some embodiments, the method further includes administering an adjuvant to the subject. In some embodiments, the method further includes administering a vaccine to the subject. The vaccine may comprise an HIV vaccine. With administration of the immunogenic composition detailed herein, the vaccine may be retained in the lymph nodes for at least 6 hours, at least 12 hours, at least 36 hours, or at least 48 hours. Administration the immunogenic composition detailed herein may boost germinal center reactions, thereby promoting a more robust immune response to the vaccine in the subject compared to a control. In some embodiments, the subject produces more IL-4 producing T cells after administration of the immunogenic composition, compared to a control. In some embodiments, the subject produces more IFNγ producing T cells after administration of the immunogenic composition, compared to a control. In some embodiments, the subject produces more FP-binding antibodies after administration of the immunogenic composition, compared to a control. The immunogenic composition detailed herein may prime the immune system of the subject prior to receiving a vaccine. The subject may be compared to the control at least 1 week, at least 2 weeks, at least 3 weeks, at least 4 weeks, at least 5 weeks, at least 6 weeks, or at least 7 weeks after administration of the nanofiber. The nanofiber may be administered prior to a vaccine. For example, the nanofiber may be administered at least 1 week, at least 2 weeks, at least 3 weeks, at least 4 weeks, at least 5 weeks, at least 6 weeks, or at least 7 weeks, prior to a vaccine. The nanofiber may be administered concurrently with a vaccine. The nanofiber may be administered after a vaccine.

### ii) Methods of Inducing an Immune Response

Further provided herein are methods of inducing an immune response in a subject. The methods may include administering to the subject the immunogenic composition as detailed herein in an amount sufficient to induce an immune response in a subject. In some embodiments, the immune response is an antigen-specific immune response. Further provided herein is an antibody produced in the immune response.

Further aspects relate to a method of inducing an immune response and/or antigen-immune response in a subject comprising administering to the subject the immunogenic composition as detailed herein in an amount sufficient to induce an immune response and/or antigen-specific immunity. In some embodiments, the immune response is an antigen-specific immune response. In some embodiments, the antigen-specific immunity is temporary and/or not life-long. In some embodiments, the antigen-specific immunity is life-long. Antigen-specific immunity refers to an adaptive immune response that occurs upon subsequent encounter with an antigenic determinant. In life-long immunity, vaccination protects the subject from environmental encounters with the antigen by inducing an immune response after the antigen has been encountered. Aspects relate to embodiments in which the immunity is temporary or lasts less than 20, 15, 10, 9, 8, 7, 6, 5, 4, 3, 2, or 1 years (or any derivable range therein). In some embodiments, the immune response comprises IgG1 antibody isotypes. In some embodiments, IgG1 antibody isotypes are the dominant antibody isotype produced in the immune response. In some embodiments, IgG1 antibody isotypes are significantly more in relation to the other antibody isotypes in the immune response. In some embodiments, the titer of IgG1 is at least 1, 1.5, 2, 2.5, or 3 log 10 units higher than other isotypes.

Further aspects relate to an antibody produced in the immune response of the methods as detailed herein.

Further methods relate to a method of treating a subject having or at risk of developing a microbial infection or viral infection or pathological condition, the method comprising administering to the subject an effective amount of a composition or antibody as detailed herein. In some embodiments, the pathological condition is cancer. In some embodiments, the pathological condition is an autoimmune disorder. In some embodiments, the pathological condition is a viral infection.

Further aspects relate to a method for making the compositions as detailed herein comprising mixing self-assembling peptides and a carrier to make a peptide fibril.

iii) Treatment Of Disease

Further provided herein are methods of treating a subject having or at risk of developing a microbial infection or viral infection or pathological condition. The methods may include administering to the subject an effective amount of a composition as detailed herein.

Embodiments relate to treatments, such as vaccines for treating cancer. In some embodiments, the cancer is glioblastoma. In some embodiments, the immune response is an anti-cancer immune response. The cancers amenable for vaccination according to the methods described herein include, but are not limited to, tumors and cancers of all types, locations, sizes, and characteristics. The methods and compositions as detailed herein are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

Embodiments can be used to treat or ameliorate a number of immune-mediated, inflammatory, autoimmune, or autoimmune-inflammatory diseases, for example, allergies, asthma, diabetes (for example, type 1 diabetes), graft rejection, etc. Examples of such diseases or disorders also include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenia purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (lgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (for example, benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, for example, due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolysis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

In some embodiments, the compositions and methods detailed herein may be used to treat bacterial infections. Bacterial infections and their related diseases may include, for example, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. In some embodiments, the bacterial infection is gonorrhea (Neisseria gonorrhea), syphilis, chlamydia, Escherichia coli, Staphylococcus spp. such as Staphylococcus aureus, Streptococcus spp such as Streptococcus pyogenes, Pseudomonas spp. such as Pseudomonas aeruginosa, or Enterococcus spp. The bacteria may be Gram positive or Gram negative. Gram-positive bacteria may include, but are not limited to, Gram positive Cocci (for example, Streptococcus, Staphylococcus, and Enterococcus). Gram-negative bacteria may include, but are not limited to, Gram negative rods (for example, Bacteroidaceae, Enterobacteriaceae, Vibrionaceae, Pasteurellae, and Pseudomonadaceae).

In some embodiments, the pathological condition is cancer or autoimmunity. In some embodiments, the pathological condition is HIV, AIDS, inflammatory bowel disease, gonorrhea, and/or rheumatoid arthritis.

### 3. Examples

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Materials and Methods

### Peptide Synthesis and Purification.

Peptides were synthesized by microwave-assisted solid phase synthesis with Fmoc-protected amino acids using a CEM Liberty Blue synthesizer, then cleaved and purified as previously described (Fries, C. N. et al. Adv. Mater. 2020, e2003310, doi:10.1002/adma.202003310, incorporated herein by reference). To generate branched peptides, Fmoc-Lys (Fmoc)-OH residues were introduced to generate multiple reactive amines for chain lengthening. To extend peptides at multiple branching points, the stoichiometry of amino acids, Oxyma and DIC were doubled at each branching point to provide sufficient reagents for multiple chain extensions. After synthesis, branched peptides were cleaved and purified similarly to linear peptide variants. To generate KLH immunogens, FP-(EAAAK)₂-C was coupled to maleimide activated Imject-KLH according to manufacturer instructions (Thermo Fischer, Waltham, MA).

### Nanofiber Formation and Atomic Force Microscopy.

Nanofibers were formed by mixing 50% FP-Coil29 with 50% Coil29 or 10% Glyco₄-Coil29 and 40% Coil29, depending on the formulation. Peptides are mixed as dry powders and dissolved in acetate buffer or solutions of Coil29Caps in acetate buffer at 8 mM total peptide concentration, as previously described (Fries, C. N. et al. Adv. Mater. 2020, e2003310, doi:10.1002/adma.202003310, incorporated herein by reference). Prior to imaging or use for immunization, peptides were diluted to 2 mM in water and 10×PBS to yield a final solution of 1×PBS. Prior to AFM imaging, nanofibers are diluted 10-fold in ultrapure water and deposited on mica substrates, then rinsed with water and dried under a stream of nitrogen. AFM imaging was completed using a Bruker AFM operated in tapping mode with Bruker RTESPA-300 silicon tips.

### Viscometry of Nanofiber Assemblies.

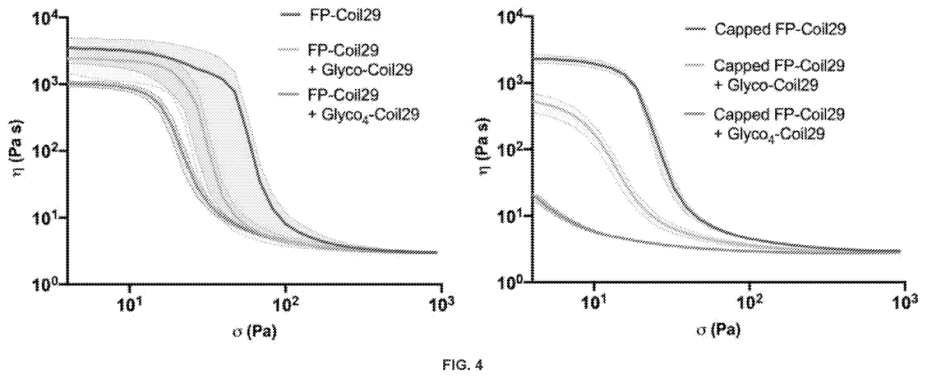

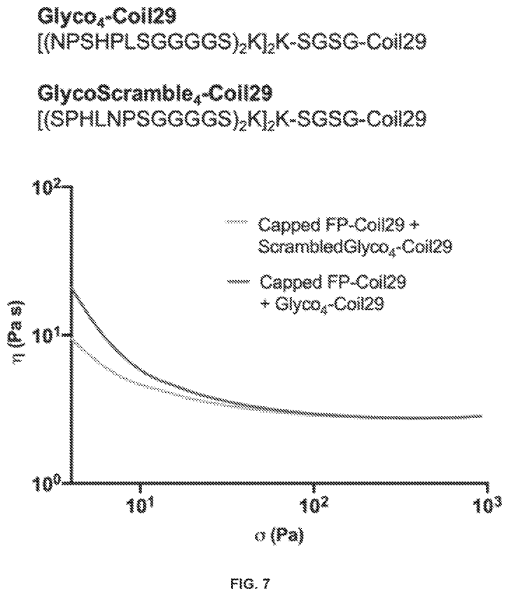

The viscosity of nanofiber solutions was utilized using a Malvern Kinexus rheometer. Frequency was ramped from 0.1-1000 Hz and sheer stress and viscosity were calculated to produce the curves shown in FIG. 4 and FIG. 7.

### Animals and Immunizations.

To study immune responses to FP-Coil29 and FP-KLH immunogens, 8-week-old C57BL/6 mice were injected subcutaneously at the tail base. For nanofiber immunizations, 2 separate 50 μL injections of 2 mM peptides were given. KLH was administered at a 10 μg dose to match previous studies, and CH505.TF SOSIP was given at a 10 μg dose after FP priming. For CpG adjuvanted immunizations, 10 μg of CpG was mixed with nanofibers, KLH or SOSIP prior to immunization. To analyze draining lymph node responses, inguinal lymph nodes were dissected and imaged by IVIS or processed for flow cytometry. For monitoring antibody responses, blood was collected from the submandibular vein at a biweekly frequency. All animal procedures were approved under IACUC protocol A264-18-11 at Duke University.

### IVIS Imaging of Lymph Nodes.

To detect nanomaterial accumulation in lymph nodes, inguinal nodes were dissected post-injection and imaged in a Perkin Elmer Lumina III with an excitation laser at 640 nm and detection at 670 nm. Radiant efficiency in nodes was then quantified using Living Image software and reported as the percentage of radiant efficiency of control lymph nodes harvested from naive mice.

### Flow Cytometry of Lymph Node Isolates.

1-2 weeks after the final boost, inguinal lymph nodes were harvested from mice and crushed before filtering through a 70 μm cell strainer. Lymphocytes were then stained and gated to detect B220+CD19+GI7hi germinal center B cells.

For fluorescent labeling of antigens for flow cytometry, and to detect gp120-specific B cells by flow cytometry, fluorescently labeled 1086.C gp120 was synthesized for cell staining. Avi-tagged 1086.C gp120 was biotinylated using BirA biotin-protein ligase standard reaction kit (Avidity Biosciences, San Diego, CA; BirA500) following the manufacturer's protocol. Tetramers were prepared based on the molar ratio (4:1) of the analyte protein and fluorochrome-conjugated streptavidin, respectively. Alexa Fluor 647 (ThermoFisher, Waltham, MA; 21374) or Brilliant Violet 421 (BioLegend, San Diego, CA; 05225) conjugated streptavidin was reacted with the biotinylated protein over 5 additions, incubating for 15 minutes between each addition. The final concentration of tetramer was calculated with respect to the analyte protein after which PBS was added to achieve concentration of 3 μM. The solution was aliquoted, snap frozen, and stored at −80° C.

For flow cytometry of lymph node-isolated cells, harvested mouse inguinal, brachial, and axillary nodes were harvested by unblinded researchers and gently ground against a 70 μm Falcon cell strainer (VWR international LLC, Radnor, PA) to break down the tissue, followed by purification using Lympholyte-M Cell Separation Media (Cedarlane, Burlington, Canada). Lymphocyte density and viability were measured by MUSE Cell Analyzer (Sigma-Millipore, St. Louis, MO). For B cell phenotyping, 2×10⁶ total calls were stained with fluorescently labeled antibodies IgG1 FITC, IgG2 FITC, IgG3 FITC, CD93 PE-CF954, IgM PE-Cy7, CD19 APC-R700, CD95 BV605, B220 BV650, CD138 BV711, CD23 BV786, and IgD BV510 (all from BD Biosciences, Franklin Lakes, NJ), GL7 PE, CD21 PerCP-Cy5.5, CD11b BV570 (both from BioLegend, San Diego, CA), CD38 PE-Cy5 (eBioscience, ThermoFisher, Waltham, MA). For T cell phenotyping, 1×10⁶ lymphocytes were stained with a panel of antibodies including CD4 FITC, CD25-PE, CD279 PE-CF594, CD62L PE-Cy7, CXCR5 Biotin, CD8a APC-R700, CD127 BV421, CD3e BV510, CD90.2 BV605, CD44 BV711, and B220 BV786 (all from BD Biosciences, Franklin Lakes, NJ), NK1.1 BV650, CD11b BV570, and CD49b PerCP-Cy5.5, and TER119 PE-Cy5 (all from Biolegend, San Diego, CA), followed by incubation with Streptavidin AF647 (Invitrogen) for CXCR5 staining. After washing with PBS, cells stained with the B-cell or T-cell panel were incubated with Live/Dead near-IR dye (Invitrogen, Waltham, MA) in 1:1000 dilution. Cells were then fixed with 2% formaldehyde in PBS and analyzed using a BD LSRII flow cytometer (BD Biosciences, Franklin Lakes, NJ). Analysis of specific cell populations was executed following a gating scheme.

### ELISPOT Detection of Antigen-Specific T Cells.

ELISPOT was used to detect peptide-responsive T cells as previously described (Wu, Y. et al. Biomater. Sci. 2020, 8, 3522-3535, doi:10.1039/d0bm00521e, incorporated herein by reference). Briefly, spleens were harvested from mice 1-2 weeks after the final immunization. Spleens were crushed and filtered through a 70 μm cell strainer and washed in cold PBS with 2% FBS. Red blood cells were then lysed with ACK lysing buffer, cells were washed, and lymphocytes were isolated using Lympholyte M. 2.5×106 cells were then mixed with stimulating peptides (5 μM) and cultured on membrane plates coated in IL-4 or IFNγ capture antibodies for 48 hours. After culture, cells were removed, and plates were washed in PBST before adding the corresponding detection antibodies to IL-4 and IFNγ plates for 2 hours at room temperature. Plates were then washed again and treated with streptavidin-ALP before a final wash and treatment with SigmaFast substrate. Plates were then monitored for the appearance of spots and washed with ultrapure water to stop spot formation. Spot counts were quantified by ZellNet Consulting for analysis.

### Elisa Analysis of Serum Antibodies.

For N-terminal and C-terminal FP fusions, Corning 96-well high binding plates were coated overnight with streptavidin (2 μg/mL) before washing and applying biotinylated FP peptides (all sequences listed in TABLE 1) at 20 μg/mL or PBS to alternating columns and incubating for 1 hour. After washing, plates were blocked with SuperBlock and murine serum diluted in 1% BSA in PBST was applied to a peptide-coated and PBS column for dilutions spanning 1:100 to 1:10,000 for 2 hours. After serum incubation, plates were washed, and murine-IgG HRP antibody was applied for 45 minutes. Finally, plates were washed, and bound antibody was measured using TMB and a phosphoric acid stop solution.

For directly coated FP ELISAs, the same method described above was followed except that FP peptides or PBS were coated overnight at 20 μg/mL before washing and blocking the following day.

To detect SOSIP-binding antibodies, plates were coated overnight in PGT145 (4 μg/mL), washed, and blocked with SuperBlock for 1 hour. SOSIP was then applied (10 μg/mL) and incubated for 2 hours at room temperature. After washing, murine serum diluted in 1% BSA in PBST was applied to wells at dilutions spanning 1:100 to 1:10,000 for 2 hours. After serum incubation, plates were washed, and murine-IgG HRP antibody was applied for 45 minutes. For all ELISAs, plates were washed, and bound antibody was measured using TMB and a phosphoric acid stop solution. To detect antibody binding, plates were read using a SpectraMax plate reader (450 nm).

### Surface Plasmon Resonance Measurement of Serum Antibodies.

To measure the abundance and binding strength of antibodies to FP, IgG was purified from serum using IgG purification columns (Thermo Fischer, Waltham, MA) and all samples were diluted to equal concentrations in PBS. Streptavidin coated SPR chips were then modified with biotin-FP and serum was flowed across chips to detect association, followed by PBS to measure dissociation rates. Dissociation curves were then fit to generate off-rate constants Ka (1/s).

### Example 2

### Design of Fusion Peptide Nanofiber Immunogens

Peptide nanofibers can raise antibody responses to peptide epitopes, with a particular capacity to generate responses towards epitopes with rare and low affinity B cell precursors. Because these nanofibers can be formed from chemically defined peptides with any given sequence, we generated FP-nanofibers using the self-assembling backbone of Coil29, a coiled-coil peptide which forms nanotubes useful for generating antibody and CD8+ T cell responses (Egelman, E. H. et al. Structure 2015, 23, 280-289, doi: papers3://publication/doi/10.1016/j.str.2014.12.008; Wu, Y. et al. ACS Biomater. Sci. Eng. 2017, 3, 3128-3132, doi:10.1021/acsbiomaterials.7b00561, each incorporated herein by reference). Coil29 can improve antibody affinity and magnitude relative to immunogens based on beta-sheet nanofiber backbones because it is highly multivalent and contains multiple T cell epitopes which generate T follicular helper responses in germinal centers. We investigated vaccines created by arraying FP peptides on highly immunogenic Coil29 carriers, and we exploited the modular nature of these materials to investigate the effect of nanofiber size and lectin binding capacity on immune responses.

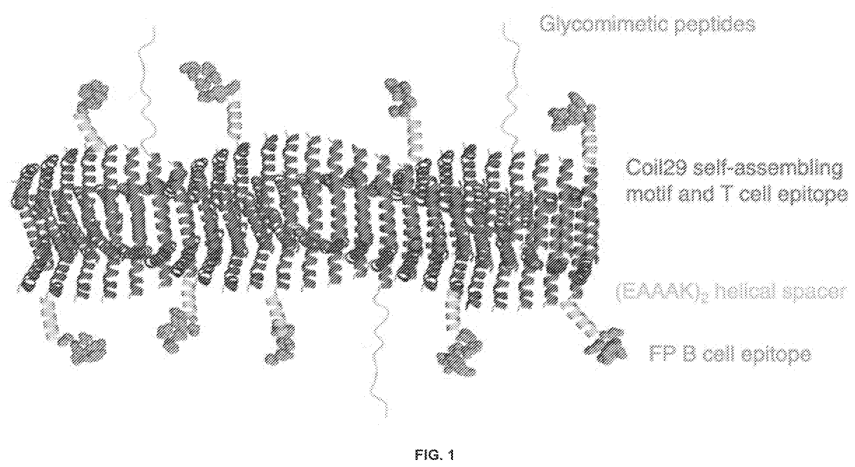

Glycosylation is important for trafficking nanomaterials to germinal centers (GCs). Increased accumulation of nanomaterials in germinal centers improves the number and affinity of B cells within GCs, effects which would likely be beneficial for establishing a dominant FP-directed response in heterologous immunization strategies. The role of glycans in promoting material trafficking to GCs is restricted to materials of a certain size, however, as these materials must drain freely to lymph nodes to be transported to B cell follicles. We sought to simultaneously minimize nanofiber size and include glycomimetic motifs to promote trafficking of materials to germinal centers. Carbohydrates of specific structures are often difficult to chemically conjugate, are sensitive to structural changes depending on pH, and specific chemically modified derivatives can often be costly. Because of these reasons, we explored the use of glycomimetic peptides as alternatives to carbohydrate modification of peptides (Eggink, L. L., et al. PLOS ONE 2015, 10, e0130532, doi:10.1371/journal.pone.0130532, incorporated herein by reference). In other studies, we developed capping peptides that were used to minimize nanofiber size improved CD8 T cell responses, potentially by improving their delivery to lymph nodes (Fries, C. N. et al. Adv. Mater. 2020, e2003310, doi:10.1002/adma.202003310, incorporated herein by reference). In this study, we incorporated these capping peptides along with glycomimetic peptides to generate size-controlled, glycomimetic immunogens composed entirely of synthetic peptides as shown in FIG. 1.

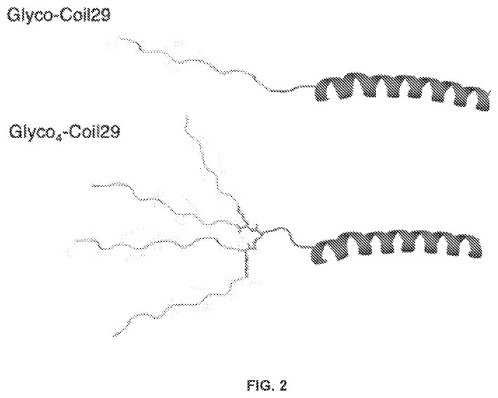

The glycomimetic peptides utilized in this study were based on a sequence that was predicted in silico and experimentally validated for binding to various lectins (Laura L. Eggink, J. K. H. Glycobiology Insights 2010, 2, 63-74, incorporated herein by reference), including CD169 (Eggink, L. L., et al. PLOS ONE 2015, 10, e0130532, doi:10.1371/journal.pone.0130532, incorporated herein by reference), a receptor expressed on macrophages which transport particulate materials to B cell follicles (Phan, T. G., et al. Nat. Immunol. 2009, 10, 787-794, doi: papers3://publication/doi/10.1038/ni.1745, incorporated herein by reference). In addition to the compelement-mediated transport of glycosylasted materials mentioned above, we hypothesized that increasing associatation of our materials with CD169 macropahges could alter the affinity maturation of B cells encountering immunogens via this pathway. The initial discovery of this CD169-binding peptide sequence determined that displaying this peptide in a tetrameric format improved its binding to CD169 receptors by increasing its avidity (Eggink, L. L., et al. PLOS ONE 2015, 10, e0130532, doi:10.1371/journal.pone.0130532, incorporated herein by reference). We synthesized tetrafunctional peptide variants of this sequence by introducing branching lysines after the linear Coil29 sequence in addition to synthesizing a monofunctional variant. The structures of both monofunctional and tetrafunctional peptides are displayed in FIG. 2.

### Example 3

### Biophysical Properties of Nanofibers Incorporating Branched and Glycomimetic Peptides

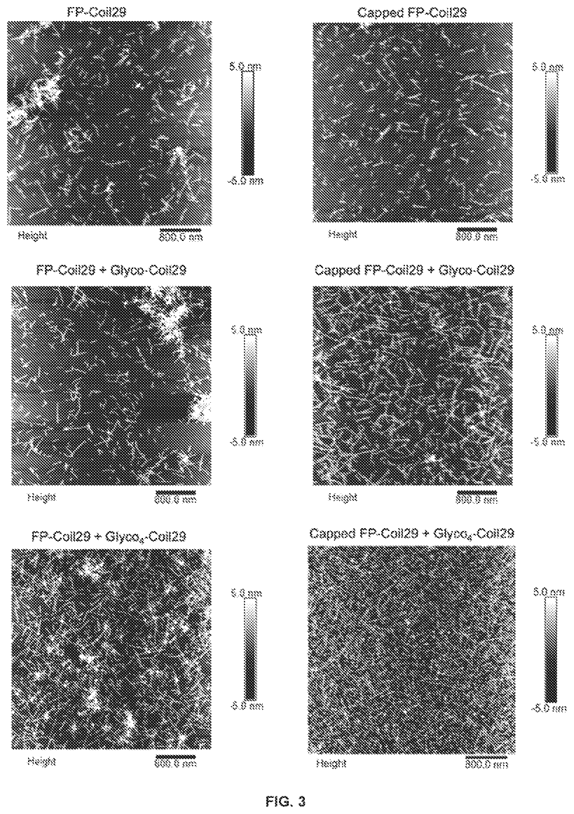

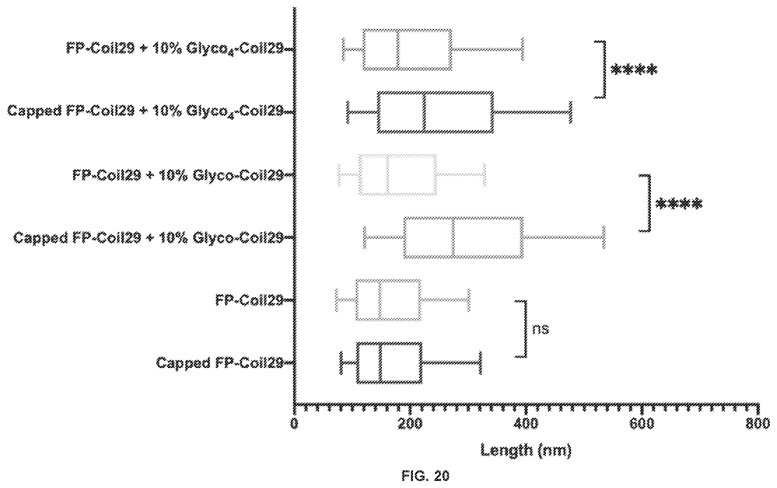

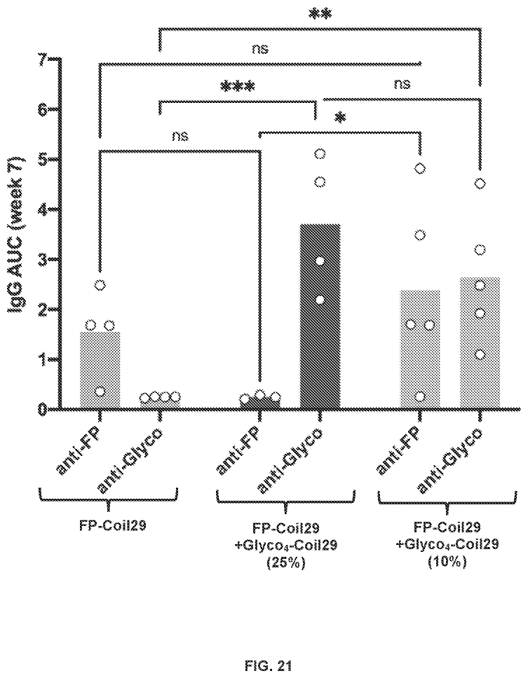

Utilizing the Coil29 backbone to assemble multiple peptides into nanostructures, we generated formulations of FP-Coil29 and glycomimetic peptides with or without capping peptides. Each of these formulations formed nanofibers as imaged by AFM as shown in FIG. 3. Notably, we observed nanofiber aggregates more commonly in samples without capping peptides, whereas capped nanofibers tended to produce nanofibers which were less likely to exist in aggregates. We also observed a phenomenon in which capping peptides generated slightly longer nanofibers than uncapped formulations containing Glyco-Coil29 or Glyco₄-Coil29 (FIG. 20). The observed effect may be due to the large proportion of aggregated material in uncapped nanofibers, leaving only short fibers soluble for adherence to mica substrates. It is also possible that Capping peptides act as nucleation sites for nanofiber assembly, leading to longer fibers because of the stable formation site created along the hydrophobic faces of Coil29Caps. Though this phenomenon was not observed for other Coil29 peptides tested in previous studies, different effects may be at play when a hydrophobic epitope such as FP is introduced into assemblies, as has been observed for high proportions of SIINFEKL-Coil29.

The presence of nanofiber aggregates noted by AFM were confirmed when analyzing the solution phase properties of nanofiber solutions using viscometry as shown in FIG. 4. In nanofiber formulations formulated without capping peptides, the inclusion of glycomimetic peptides did not significantly alter solution properties. In capped formulations, however, glycomimetic peptides reduced the viscosity or nanofibers (Capped FP-Coil29+Glyco-Coil29) and produced markedly less viscous nanofibers when incorporated in a branched format (Capped FP-Coil29+Glyco₄-Coil29).

We interpreted this change in viscosity as a likely indication that both branched Glyco₄-Coil29 peptides and Capping peptides are necessary to generate a solution of nanofibers which are not associated with each other in a stiff network. This could be attributed to the possible existence of brush-like conformations of branched peptides which could act to repel nanofibers from each other. However, this phenomenon only appears to be at play when nanofibers are sufficiently stabilized at their tips by capping peptides, allowing stable monomeric nanofibers to form. Though we expected to observe a similar reduction of viscosity for uncapped nanofibers containing Glyco₄-Coil29, this formulation showed a similar viscosity profile to other uncapped nanofibers. As mentioned above, this could be due to the more common existence of aggregated nanofibers, potentially because assembly is not as efficient without capping peptides providing nucleation sites, stabilizing the tips of formed nanofibers, or serving both of these functions.

### Example 4

### Accumulation of Nanomaterials in Draining Lymph Nodes

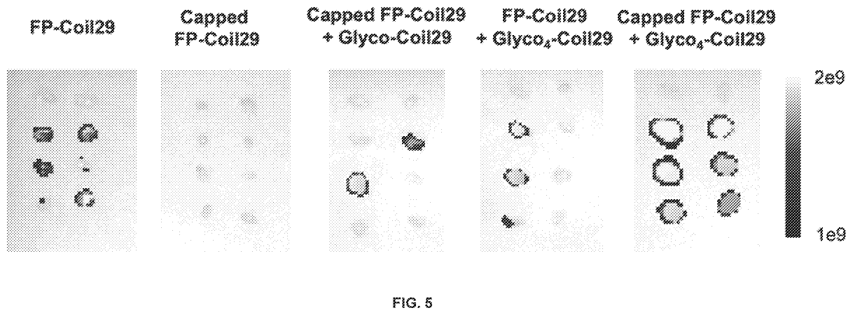

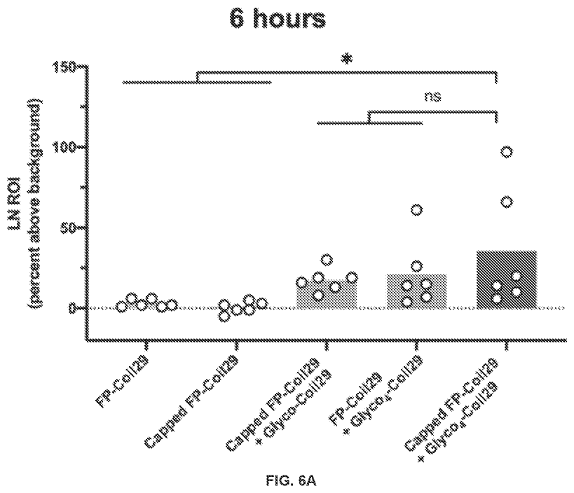

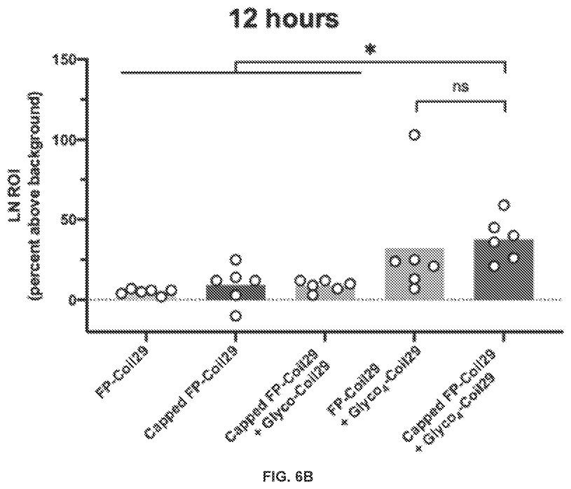

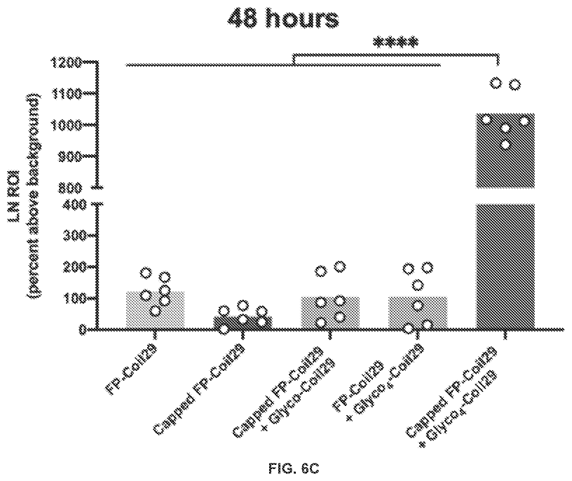

Because nanomaterial size largely dictates drainage to lymph nodes and material capture and accumulation is influenced by glycosylation, we expected nanofiber capping and glycomimetic peptide content to each have an influence on the accumulation of our nanofibers in lymph nodes. In most cases, nanomaterials <100 nm in diameter drain freely to lymph nodes while materials >100 nm in diameter require cell mediated transport. Most of these trafficking studies have been completed on spherical liposomes or polystyrene particles, but there is a limited understanding of how nanofiber size changes lymphatic trafficking. We incorporated Coil29 peptides fluorescently labeled with AlexaFluor647 into nanofiber assemblies and measured their accumulation in draining lymph nodes after subcutaneous injection. Inguinal lymph nodes were excised at 6, 12, and 48 hours after tail base injection and measured via IVIS to quantify the amount of material present in each node as shown in FIG. 5 and FIG. 6.

We detected the presence of fluorescent material earlier in the lymph nodes of mice which were injected with nanofibers containing glycomimetic peptides regardless of capping peptide content, suggesting that the glycomimetic peptide sequence promoted retention of nanofibers in lymph nodes after passive drainage. After 48 hours, when both active and passive transport play a role in nanomaterial trafficking, we detected a striking increase in material for Capped FP-Coil29+Glyco₄-Coil29 relative to all other nanofiber formulations. This suggested that both glycomimetic peptide content and unaggregated-nanofiber solution properties are critical for promoting lymphatic trafficking and retention. Because of this improvement in nanofiber trafficking, we explored the use of branched Glyco₄-Coil29 in FP-directed immunization strategies.

### Example 5

### Glycomimetic Peptide Sequence Promotes Accumulation in Lymph Nodes and Boosts Germinal Center Reactions

We observed differences in the biophysical properties of Capped glycomimetic nanofibers, and we asked whether the reduction of nanofiber aggregation was responsible for improved lymph node accumulation, or if this effect was dependent on the glycomimetic peptide sequence we selected for this study. To test the influence of glycomimetic peptide sequences, we generated a branched peptide with a scrambled glycomimetic sequence shown in FIG. 7. Capped nanofibers made with scrambled and unscrambled glycomimetic sequences displayed similar viscometry profiles (FIG. 7).

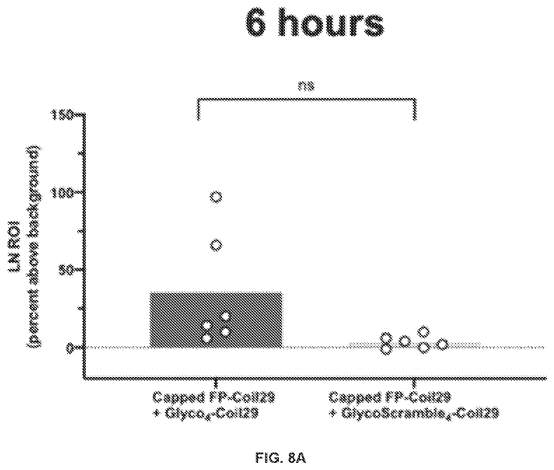

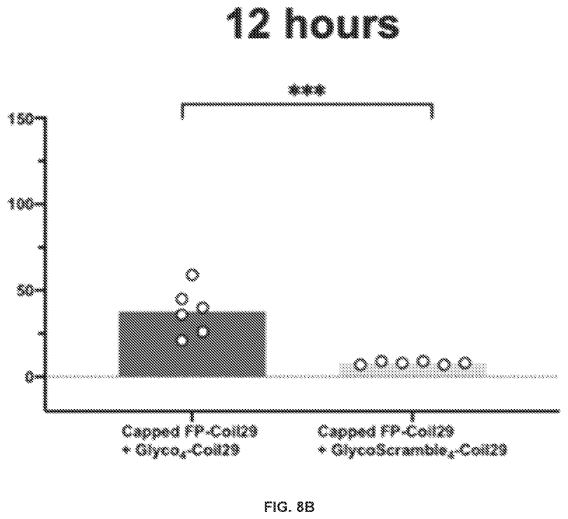

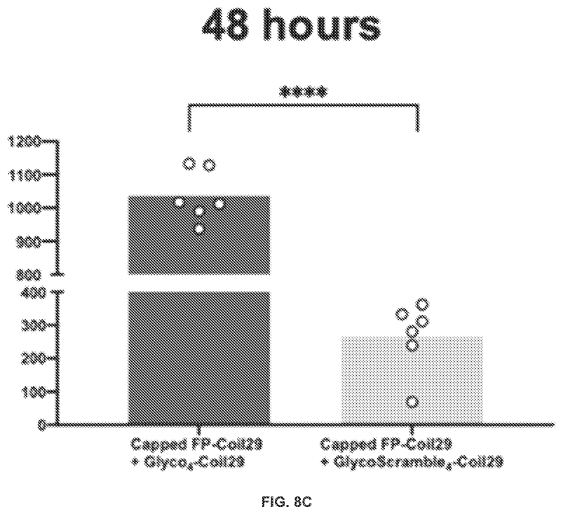

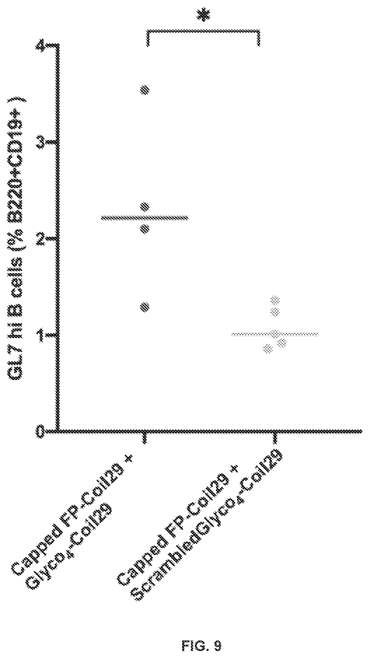

Despite having similar biophysical properties and containing branched peptides with the same amino acid composition, the Siglec-binding glycomimetic sequence showed a strong preference for lymph node accumulation compared to its scrambled comparison as measured by IVIS and shown in FIG. 8. After 12-hours and 48-hours post-injection, the glycomimetic sequence increased accumulation of fluorescently labeled nanofibers in lymph nodes, indicating that the lectin-binding properties of this sequence are key to its activity.

To determine the immunological influence of glycomimetic sequences on immune responses, we immunized C57BL/6 mice with 3 doses of Capped nanofibers incorporating branched peptides with a lectin-binding or scrambled glycomimetic sequence. After the final immunization, the draining lymph nodes of mice which received the Glyco₄-Coil29 formulation produced more germinal center B cells than a scrambled GlycoScramble₄-Coil29 immunization. Because of the improved trafficking and germinal center responses we observed for Capped FP-Coil29+Glyco₄-Coil29 immunizations, we analyzed their activity as immunogens for priming responses to FP.

### Example 6

### Antibody and T Cell Responses to Fusion Peptide Immunogens

After observing the influence of nanofiber size and glycomimetic character on LN accumulation and germinal center reactions, we hypothesized that Capped FP-Coil29+Glyco₄-Coil29 might produce stronger T cell and antibody response to after FP immunizations than uncapped or non-glycomimetic formulations.

### Antibody Responses to Fusion Peptide Immunogens.

Mice were immunized with FP-Coil29 nanofibers including either capping peptides, glycomimetic peptides, or both and compared to the peptide immunogen carrier KLH. The KLH variant used in this study utilized a rigid helical spacer (EAAAK, see TABLE 1 for all peptide sequences) to display FP which matched nanofiber displays, but differs from previously published variants which have utilize a Cysteine-maleimide linkage without additional spacer sequences (Xu, K. et al. Nat. Med. 2018, 1-19, doi: papers3://publication/doi/10.1038/s41591-018-0042-6, incorporated herein by reference), and tested heterobifunctional crosslinkers such as MBS, Sulfo-SIAB, and PEG to link FP to carrier proteins (Ou, L. et al. Sci. Rep. 2020, 10, 3032, doi:10.1038/s41598-020-59711-y, incorporated herein by reference). The inclusion of an (EAAAK) x spacer sequence between FP and Coil29 greatly improved its solubility and formed nanofibers more readily than peptides using a short flexible linker (SGSG), so we utilized this linker in all constructs examined in this study.

TABLE 1
FP-Coil29 Sequences.

| | |
|---|---|
| FP-Coil29 | AVGIGAVFL-SGSG-(EAAAK)₂-SGSG |
| | QARILEADAEILRAYARILEAHAEILRAQ-NH₂ |
| | (SEQ ID NO: 38) |
| Alexa647- | AlexaFluor⁶⁴⁷-SGSG-(EAAAK)₂-SGSG |
| Coil29 | QARILEADAEILRAYARILEAHAEILRAQ-NH₂ |
| | (SEQ ID NO: 39) |
| Coil29 | Ac-QARILEADAEILRAYARILEAHAEILRAQ- |
| | NH₂ (SEQ ID NO: 35) |
| Coil29Cap | Ac-QSRITEADSEITRAYSRITEAHSEITRAQ- |
| a/d | NH₂ (SEQ ID NO: 32) |
| Coil29Cap | Ac-QARTLESDAETLRSYARTLESHAETLRSQ- |
| c/f | NH₂ (SEQ ID NO: 34) |
| Biotin-FP | Biotin-SGSG-AVGIGAVFL |
| | (SEQ ID NO: 40) |
| FP-Biotin | AVGIGAVFL-SGSG-(EAAAK)₂-SGSG-C- |
| | PEG-biotin |
| | (SEQ ID NO: 41) |
| FP | AVGIGAVFL (SEQ ID NO: 28) |

PEG = polyethylene glycol, FP epitope, Coil29 assembling sequence, Ac = acetyl.
The peptides above are listed from the N-terminus to the C-terminus, left-to-right.
The Coil29 peptides include acetylated N-terminus (Ac) on the left and amidated C-terminus (NH₂) on the right.

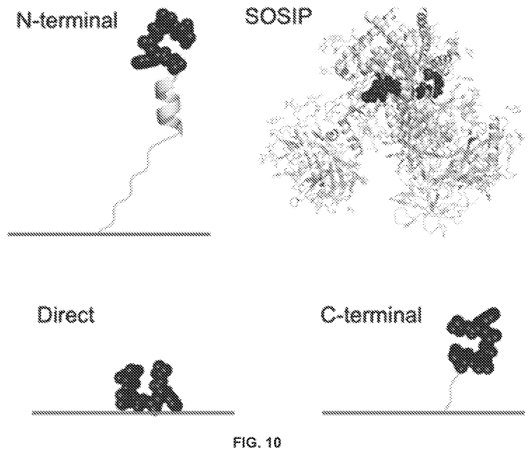

After immunization, all Coil29 scaffolded FP immunogens produced FP-reactive antibodies, but they displayed different binding modes to differentially displayed FP epitopes. Because the ability of antibodies to bind conformationally distinct FP variants is important for generating neutralizing responses, we assessed antibody binding to FP via a variety of ELISA techniques in which the antigen was immobilized onto the plate in different conformations (FIG. 10). Of the ELISA methods used, the N-terminal FP fusion displayed the least stringent binding requirements for antibodies as the FP peptide is displayed in the same N-terminal format as in Coil29 and KLH immunogens and is extended from the surface of ELISA plates via a PEG linker. Directly adsorbed FP peptide and C-terminal fusions showed more stringent binding requirements, as these peptide conformations are less solvent-available than N-terminal fusions. Finally, SOSIP trimers captured by apex-binding PGT145 antibodies displayed the FP epitope in its native-like conformation.

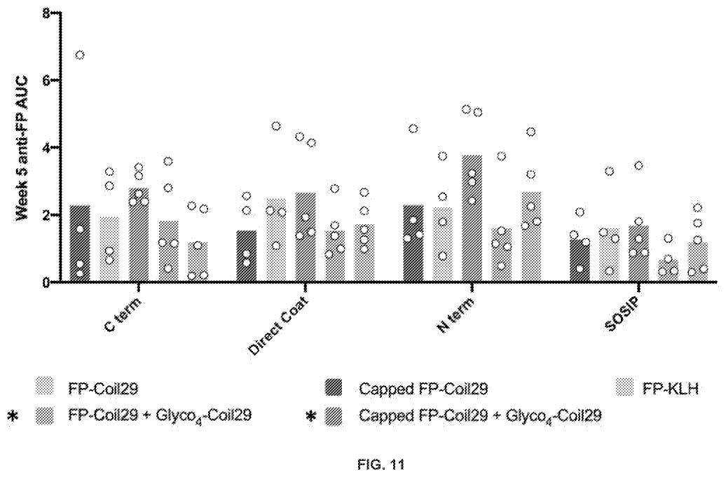

Across the coating conditions mentioned above, Capped FP-Coil29+Glyco₄-Coil29 induced a higher magnitude of antibodies which bound the diverse FP conformations displayed by each of these ELISA methods (FIG. 11). Uncapped FP-Coil29+Glyco₄-Coil29 surprisingly showed a significantly lower magnitude antibody response across these conditions. This suggested that the introduction of multiple copies of the Glyco peptide may compete with B cells' ability to bind the FP epitope, but it may be overcome by the benefits of improved LN accumulation observed for Capped FP-Coil29+Glyco₄-Coil29.

### T Cell Responses to Fusion Peptide Immunogens.

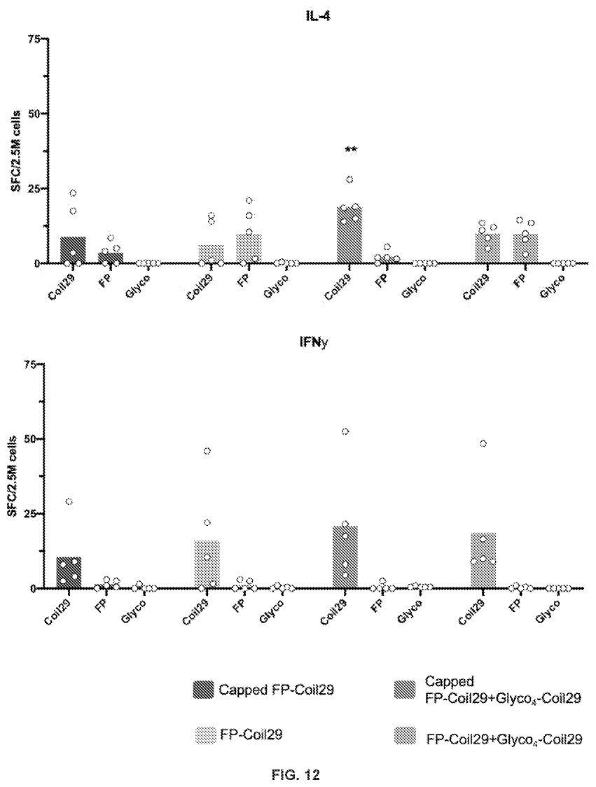

We also analyzed T cell responses to the FP-Coil29 immunogens to determine if LN accumulation also impacted the extent of T cell help induced by these formulations. After 3 immunizations, spleens were harvested from immunized mice and stimulated with immunizing peptides for analysis by ELISPOT. The self-assembling Coil29 sequence contains multiple T cell epitopes which increase its immunogenicity in mice (Wu, Y., et al. Biomater. Sci. 2020, 8, 3522-3535, doi:10.1039/d0bm00521e, incorporated herein by reference) and Coil29 reactive T cells were detected in the splenocytes of all mice immunized with FP-Coil29 nanofibers (FIG. 12). We observed an increase in Coil29-reactive IL-4 producing T cells for mice immunized with Capped FP-Coil29+Glyco₄-Coil29 which is consistent with the increased nanofiber drainage to the lymph nodes of these mice.

Because Capped FP-Coil29+Glyco₄-Coil29 immunizations showed promising features in preliminary work as singular immunogens, we were extremely interested in their use as priming immunogens for priming responses to HIV trimers.

### Example 7

### Use of Glycomimetic Immunogens to Prime Epitope-Specific B Cells

To initiate lineage-directed responses to HIV trimers, immunogens are delivered sequentially to target specific sites on the HIV trimer (Xu, K. et al. Nat. Med. 2018, 24, 857-+, doi:10.1038/s41591-018-0042-6; Yun, D. S. et al. Immunity 2016, 45, 483-496, doi: papers3://publication/doi/10.1016/j.immuni.2016.08.016, each incorporated herein by reference). In the case of fusion peptide-directed responses, multiple doses of FP carrier proteins are administered before boosting with native-like SOSIP trimers to induce a neutralizing antibody response (Xu, K. et al. Nat. Med. 2018, 24, 857-+, doi:10.1038/s41591-018-0042-6, incorporated herein by reference). To test the preclinical efficacy of FP-Coil29 nanofibers in this context, we administered 3 doses of FP-Coil29 immunogens formulated with CpG adjuvant to mice followed by 3 doses of SOSIP trimer also formulated with CpG. Throughout this regimen, we monitored antibody responses to FP in various ELISA formats and measured SOSIP binding antibodies.

### Native-Like HIV Trimers Boost Antibody Responses to Fusion Peptide Primed by Nanomaterials.

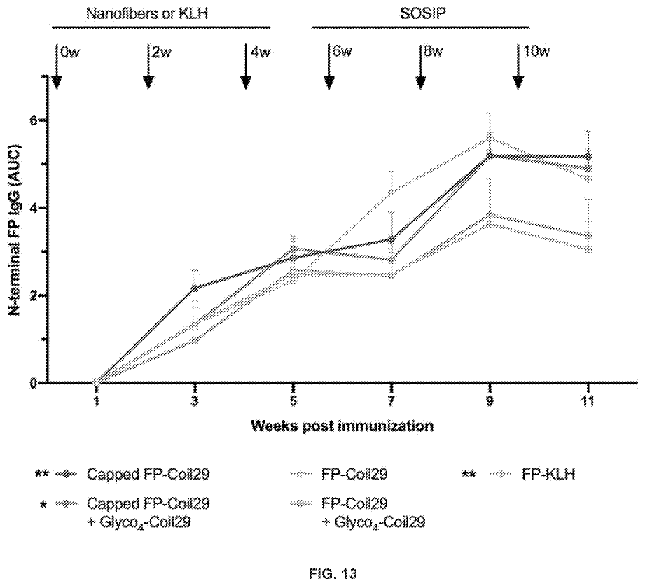

Capped and uncapped FP-Coil29 fibers with and without glycomimetic peptides were administered in comparison to FP-KLH. After 3 doses of these immunogens, all mice developed antibodies towards the N-terminally displayed FP peptide (FIG. 13). After boosting with SOSIP trimers, these antibodies continued to expand (FIG. 13), indicating that the FP B cell lineage developed by priming immunogens was also reactive to SOSIP trimers. As this regimen progressed, the magnitude of FP antibodies induced by Capped FP-Coil29, Capped FP-Coil29+Glyco₄-Coil29, and FP-KLH were greater than those induced by FP-Coil29.

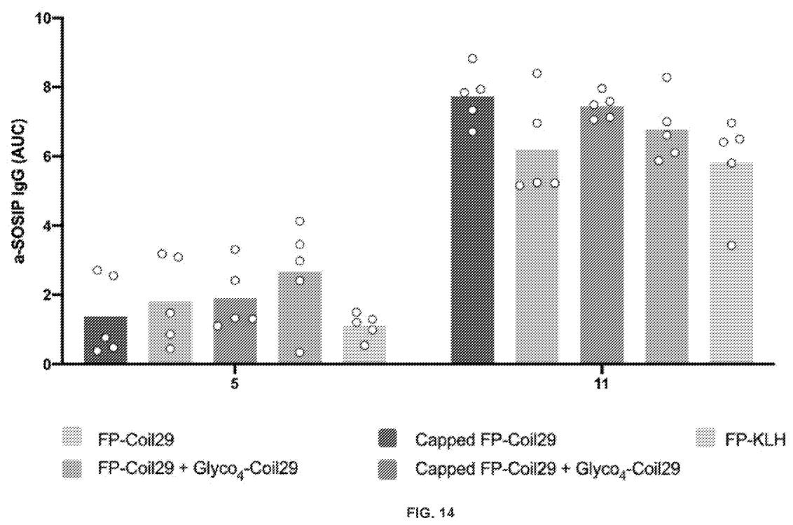

The antibodies produced after FP-directed priming also bound SOSIP by ELISA (FIG. 14), though differences in SOSIP binding were not statistically different between priming immunogens. SOSIP-reactive antibodies continued to expand throughout the immunization regimen and likely included many epitope specificities after 3 doses of SOSIP were administered.

### Priming Immunogens Shape Antibody Binding Profiles.

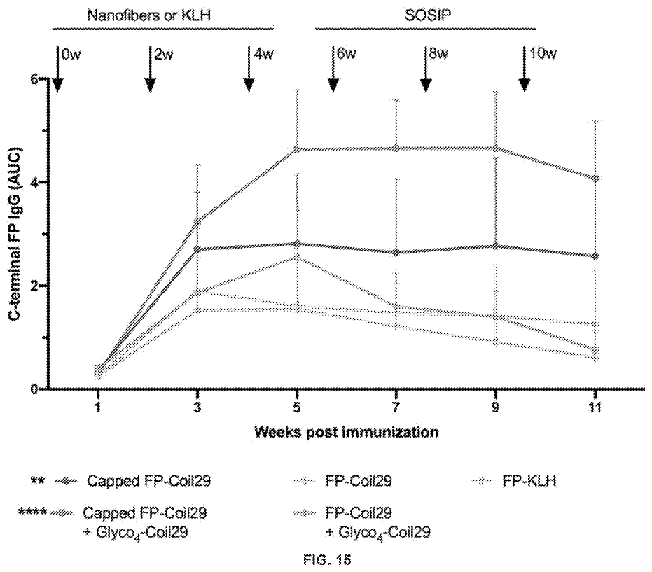

The levels of FP-binding antibodies over the course of the FP SOSIP regimen showed differential evolution as we measured antibody binding to C-terminal and directly adsorbed FP epitopes. Particularly for C-terminal FP binding antibodies, SOSIP boosting caused the decay of this antibody subset upon boosting, indicating that antibody responses evolved toward a particular conformation which was not displayed by the C-terminal FP ELISA (FIG. 15). However, mice which were primed with Capped FP-Coil29+Glyco₄-Coil29 retained high levels of C-terminal FP binding antibodies, indicating that B cells which recognize this FP conformation were not outcompeted by other B cell phenotypes in germinal centers. Mice primed with Capped FP-Coil29 also showed the persistence of C-terminal FP reactive antibodies, indicating the B cell responses induced by capped nanofiber immunogens develop unique binding properties compared to uncapped nanofibers and KLH.

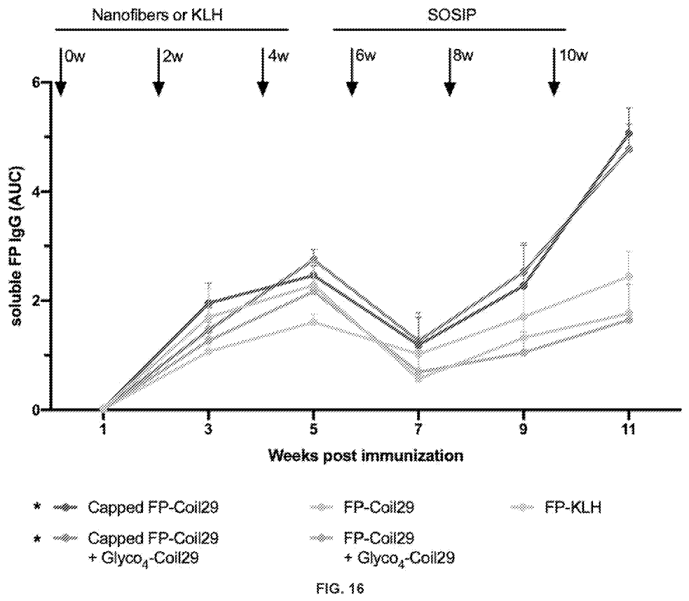

We also observed differences in antibody binding to surface adsorbed FP wherein capped nanofibers induced stronger antibodies to this FP conformation over the course of the FP SOSIP immunization regimen (FIG. 16).

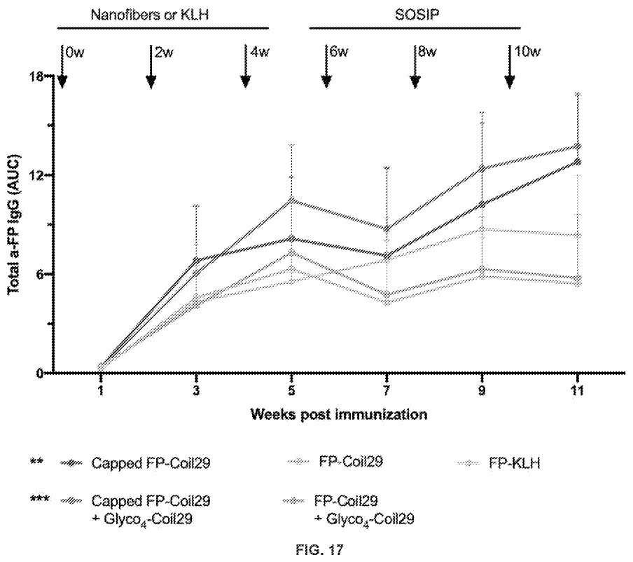

Though no single ELISA method is indicative of superior FP antibody functionality, generating an antibody response which can bind structurally diverse conformations of FP is desirable for generating neutralizing responses to FP. To analyze these responses as a whole, we added the signals from each of the three peptide ELISAs together to compare the total magnitude of FP-binding antibody responses generated by FP-Coil29 and KLH vaccines (FIG. 17). When these three binding scenarios are examined in parallel, mice which were primed with Capped FP-Coil29+Glyco₄-Coil29 display the highest magnitude FP antibodies, followed closely by mice primed with Capped FP-Coil29 (FIG. 17).

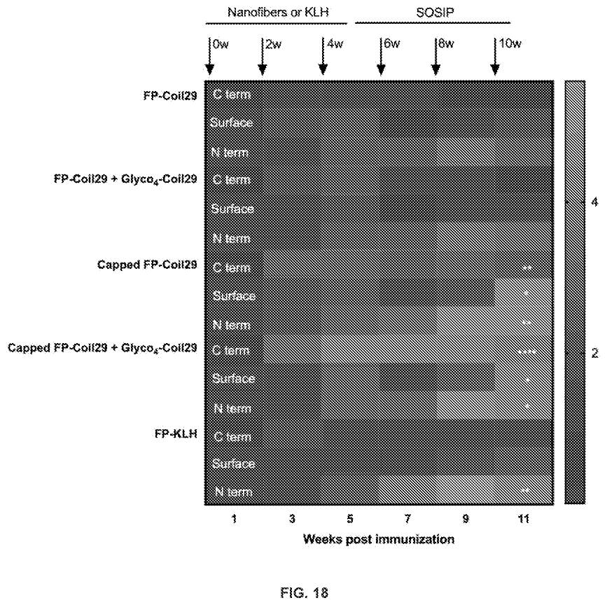

The combination of these antibody responses can also be visualized in parallel using the heatmap shown in FIG. 18 where each row displays the antibody response to a given coating over the course of the regimen for mice primed with FP-Coil29 or KLH immunogens.

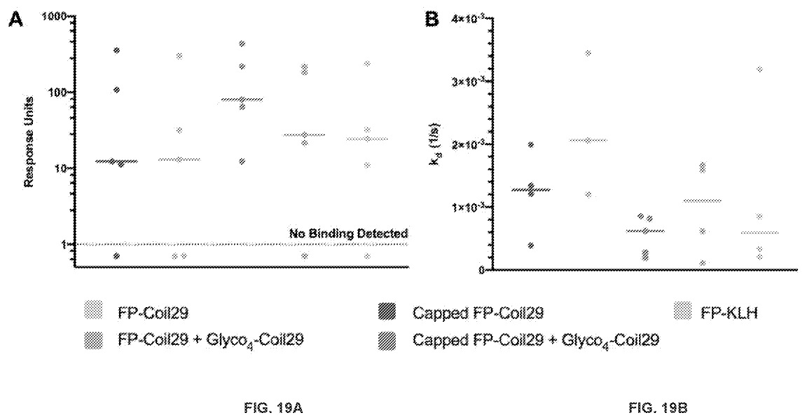

Because we measured differences in the evolution of FP antibodies across FP SOSIP boosting regimens, we hypothesized that the magnitude and affinity of antibodies after FP-immunogen priming may have influenced the fitness of FP-reactive B cells in germinal centers after introducing new epitopes upon SOSIP boosting. To measure this, we purified IgG from serum obtained after FP priming and measured binding to C-terminal FP by Surface Plasmon Resonance (SPR) as shown in FIG. 19. Though no statistically significant differences were measured between the antibodies induced by each priming immunogen, we did observe a modest trend towards a higher magnitude of FP antibodies in the serum of mice immunized with Capped FP-Coil29+Glyco₄-Coil29. The antibodies isolated from these mice also trended towards tighter binding to FP as measured by the dissociation rate constant (kd).

Taken together, the distinct FP binding antibody profiles generated by FP-Coil29 immunogens indicated the potential for improving FP-direct immunization approaches by diversifying the binding modes of FP reactive antibodies.

### Example 8

### Discussion

Self-assembling peptide vaccines are uniquely positioned to induce robust antibody responses to short peptide epitopes because of their opportunity for extremely dense epitope display, scalable synthetic production, and modular features. Though the influence of conformationally distinct antibody binding has been under-appreciated, the investigation of FP-directed immunization highlights the utility of generating antibodies which are flexible to epitope structural changes. In addition to FP-directed antibodies, the ability to generate antibodies with a broad range of binding modes could improve the ability of vaccines to induce antibodies which are adaptable to viral strains which have mutated from the antigenic variant used in immunizations. Widespread global diseases such as influenza and SARS-CoV2 may benefit from such approaches as these viruses are both widely circulating and present evolving variants over time.

Though the neutralizing capacity of antibodies was not examined in this study, the ability to generate multiple antibody binding modes toward a known neutralizing epitope effectively allows for more “shots on goal” as the immune system generates antibodies with neutralization potential. In other studies, the angle at which antibodies approach neutralizing targets has influenced their neutralizing capacity significantly, and generating vaccines which diversify these possibilities could be broadly useful for vaccinating against viruses which are traditionally difficult to neutralize.

The inclusion of branched glycomimetic peptides impacted the resultant trafficking and antibody responses toward peptide nanofibers, but the exact pathways which these peptides act on is unclear. Glycosylation has been shown to traffic nanomaterials to germinal centers via MBL-mediated complement activation, which may be at play in this system. Additionally, the glycomimetic sequences selected for this study are known to bind Siglecs, which are widely expressed among immune cells. Specifically, CD169 is a known ligand of the peptide utilized here and is expressed on subcapsular sinus (SCS) macrophages, which transport materials to B cell follicles. If the peptides on these nanofibers bind SCS macrophages in vivo, their shuttling of materials to B cell follicles could improve germinal center reactions compared to materials transported via other pathways.

In previous work, the linker used between KLH carrier proteins and FP epitopes was shown to alter the neutralizing functions of the antibodies these vaccines elicited. The helical linker used in this study was employed to provide a rigid spacer between the hydrophobic FP epitope and self-assembling Coil29 domain and may have affected the responses to FP-KLH which we observed relative to other published constructs. In comparison to KLH conjugates which have been explored in the context of FP-directed HIV immunization, peptide nanofibers offer many advantages. Coil29 peptides can be produced entirely by chemical synthesis, which allows for scalable production and chemically well-defined therapeutics. Relative to KLH, these peptide-based systems have significantly improved batch-to-batch consistency because of their facile purification and modular composition.

For deployment of an HIV-1 vaccine in limited resource settings, the need for cold-chain distribution presents significant logistical challenges. Peptide nanofibers are able to be stored as freeze-dried powders, which would provide immense benefit to the cost and timeliness of global vaccine distribution. In addition to practical advantages, nanofibers may allow for delivery across mucosal routes, which could provide enhanced protection for vaginally or rectally transmitted HIV virus.

The FP-Coil29 immunogens described here open up potential avenues for generating FP-directed antibody responses. Because of the diversified antibody binding modes elicited by Capped FP-Coil29+Glyco₄-Coil29 immunizations, these priming immunogens have significant promise for their ability to generate functional antibodies in higher order species.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:
Clause 1. A composition comprising: (i) a nanofiber comprising: a backbone comprising a plurality of self-assembling peptides, wherein each self-assembling peptide forms an alpha-helix, and wherein the self-assembling peptide comprises an amino acid sequence of bXXXb (SEQ ID NO: 1), wherein X is independently any amino acid, and b is independently any positively charged amino acid; at least one glycomimetic peptide linked to the backbone; and at least one antigen, wherein each antigen is individually linked to the backbone by a helical linker; or (ii) a nanofiber comprising: a backbone comprising a plurality of self-assembling peptides, wherein each self-assembling peptide forms an alpha-helix, and wherein the self-assembling peptide comprises an amino acid sequence of bXXXb (SEQ ID NO: 1), wherein X is independently any amino acid, and b is independently any positively charged amino acid; and at least one antigen, wherein each antigen is individually linked to the backbone by a helical linker; or (iii) a nanofiber comprising: a backbone comprising a plurality of self-assembling peptides, wherein each self-assembling peptide forms an alpha-helix, and wherein the self-assembling peptide comprises an amino acid sequence of bXXXb (SEQ ID NO: 1), wherein X is independently any amino acid, and b is independently any positively charged amino acid; and at least one glycomimetic peptide linked to the backbone; or (iv) any combination of (i), (ii), and (iii).
Clause 2. The composition of clause 1, wherein the antigen comprises an epitope for a disease selected from HIV, inflammatory bowel disease, gonorrhea, or rheumatoid arthritis.
Clause 3. The composition of clause 1 or 2, wherein bXXXb (SEQ ID NO: 1) is RAYAR (SEQ ID NO: 2) or KAYAK (SEQ ID NO: 3).
Clause 4. The composition of clause 1 or 2 or 3, wherein the backbone comprises an amino acid sequence of ZnbXXXbZm (SEQ ID NO: 5), wherein b is independently any positively charged amino acid, Z is independently any amino acid, X is independently any amino acid, n is an integer from 0 to 20, and m is an integer from 0 to 20.
Clause 5. The composition of any one of clauses 1-4, wherein the backbone comprises an amino acid sequence selected from QARILEADAEILRAYARILEAHAEILRAQ (SEQ ID NO: 6), or QAKILEADAEILKAYAKILEAHAEILKAQ (SEQ ID NO: 7), or ADAEILRAYARILEAHAEILRAQ (SEQ ID NO: 8), or SEQ ID NO: 35, or SEQ ID NO: 36, or SEQ ID NO: 37.
Clause 6. The composition of clause 5, wherein the backbone comprises the amino acid sequence of SEQ ID NO: 6 (QARILEADAEILRAYARILEAHAEILRAQ; Coil29 (PDB 3J89)).
Clause 7. The composition of any one of clauses 1-6, wherein the backbone has a coiled coil structure.
Clause 8. The composition of any one of clauses 1-6, wherein the backbone has a structure of a helical filament formed around a central axis.
Clause 9. The composition of clause 8, wherein the N-terminus of each backbone is positioned at the exterior of the helical filament.
Clause 10. The composition of any one of clauses 1-9, wherein each of the at least one glycomimetic peptide is capable of binding to a lectin.
Clause 11. The composition of clause 10, wherein the lectin comprises CD169.
Clause 12. The composition of any one of clauses 1-11, wherein the glycomimetic peptide is linked to the backbone by peptide linker.
Clause 13. The composition of clause 12, wherein the peptide linker comprises the amino acid sequence selected from SEQ ID NO: 9 (Gn wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 29 (KSGSG), SEQ ID NO: 30 (KKSGSG), and SEQ ID NO: 31 (EAAAK)₂.
Clause 14. The composition of any one of clauses 1-13, wherein the at least one glycomimetic peptide comprises a linear peptide, a branched peptide, or a combination thereof.
Clause 15. The composition of clause 14, wherein the branched peptide comprises at least 3 or 4 branches.
Clause 16. The composition of clause 14 or 15, wherein the branched peptide is linked to the backbone by peptide linker comprising at least one lysine.
Clause 17. The composition of any one of clauses 1-16, wherein the glycomimetic peptide comprises the amino acid sequence of SEQ ID NO: 25 [NPSHPLSGGGGS], SEQ ID NO: 26 [(NPSHPLSGGGGS)₂K], SEQ ID NO: 27 [((NPSHPLSGGGGS)₂K)₂], or a combination thereof.
Clause 18. The composition of any one of clauses 1-17, wherein the nanofiber comprises 2 to 10, 2 to 8, 2 to 6, or 2 to 4 glycomimetic peptides.
Clause 19. The composition of any one of clauses 1-18, wherein the antigen comprises a FP B-cell epitope.
Clause 20. The composition of clause 19, wherein the FP B-cell epitope comprises the amino acid sequence of SEQ ID NO: 28 (AVGIGAVFL).
Clause 21. The composition of any one of clauses 1-18, wherein the antigen comprises a B cell epitope in TNF, or IL-17, or phosphorylcholine, or a complement C3dg, or a B cell epitope in complement C5a, or a peptide comprising the amino acid sequence of SEQ ID NO: 45.
Clause 22. The composition of any one of clauses 1-21, wherein the nanofiber comprises 2 to 20, 2 to 15, 2 to 12, 2 to 10, 2 to 8, or 2 to 6 antigens.
Clause 23. The composition of any one of clauses 1-22, wherein the helical linker comprises a peptide comprising the amino acid sequence selected from SEQ ID NO: 9 (Gn wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), and SEQ ID NO: 31 (EAAAK)₂.
Clause 24. The composition of any one of clauses 1-23, wherein the nanofiber further comprises one or more capping peptides.
Clause 25. The composition of clause 24, wherein the capping peptide comprises an amino acid sequence selected from SEQ ID NOs: 32, 33, 34, 42, 43, 44, or a combination thereof.
Clause 26. The composition of any one of clauses 1-25, wherein the nanofiber is 50 nm to 600 nm in length.
Clause 27. A method of immunizing a subject, the method comprising: administering to the subject a therapeutically effective amount of the composition of any one of clauses 1-26.
Clause 28. The method of clause 27, further comprising administering an adjuvant to the subject.
Clause 29. The method of clause 27 or 28, further comprising administering a vaccine to the subject.
Clause 30. The method of clause 29, wherein the vaccine is retained in the lymph nodes for at least 6 hours, at least 12 hours, at least 36 hours, at least 48 hours after administration.
Clause 31. The method of any one of clauses 27-30, wherein the composition boosts germinal center reactions, thereby promoting a more robust immune response to the vaccine in the subject compared to a control.
Clause 32. The method of clause 31, wherein the subject produces more IL-4 producing T cells after administration, compared to a control.
Clause 33. The method of clause 31 or 32, wherein the subject produces more IFNγ producing T cells after administration, compared to a control.
Clause 34. The method of clause 31, 32, or 33, wherein the subject produces more antigen-binding antibodies or FP-binding antibodies after administration, compared to a control.
Clause 35. The method of any one of clauses 31-34, wherein the subject is compared to the control at least 1 week, at least 2 weeks, at least 3 weeks, at least 4 weeks, at least 5 weeks, at least 6 weeks, or at least 7 weeks after administration of the nanofiber.
Clause 36. The method of any one of clauses 29-35, wherein the vaccine comprises an HIV vaccine.
Clause 37. The method of any one of clauses 29-36, wherein the at least one nanofiber is administered prior to the vaccine.
Clause 38. The method of clause 37, wherein the at least one nanofiber is administered at least 1 week, at least 2 weeks, at least 3 weeks, at least 4 weeks, at least 5 weeks, at least 6 weeks, or at least 7 weeks, prior to the vaccine.
Clause 39. The method of clause 38, wherein the composition of any one of clauses 1-26 primes the immune system of the subject prior to receiving the vaccine.
Clause 40. The method of any one of clauses 29-36, wherein the at least one nanofiber is administered concurrently with the vaccine.
Clause 41. The method of any one of clauses 29-36, wherein the at least one nanofiber is administered after the vaccine.

SEQUENCES
SEQ ID NO: 1
bXXXb
wherein X is independently any amino acid, and b is
independently any positively charged amino acid.
SEQ ID NO: 2
RAYAR
SEQ ID NO: 3
KAYAK
SEQ ID NO: 4
RXXXR
wherein X is any amino acid.
SEQ ID NO: 5
ZₙbXXXbZₘ
wherein b is independently any positively charged amino acid,
Z is independently any amino acid, X is independently any
amino acid, n is an integer from 0 to 20, and m is an integer
from 0 to 20.
Coil29 amino acid sequence
SEQ ID NO: 6
QARILEADAEILRAYARILEAHAEILRAQ
SEQ ID NO: 7
QAKILEADAEILKAYAKILEAHAEILKAQ
Coil23 amino acid sequence
SEQ ID NO: 8
ADAEILRAYARILEAHAEILRAQ
SEQ ID NO: 9
Gₙ
wherein n is an integer from 1 to 10
SEQ ID NO: 10
SGSG
SEQ ID NO: 11
GSGS
SEQ ID NO: 12
SSSS
SEQ ID NO: 13
GGGS
SEQ ID NO: 14
GGC
SEQ ID NO: 15
(GGC)₈
SEQ ID NO: 16
(G₄S)₃
PEPvIII
SEQ ID NO: 17
LEEKKGNYVVTDH
PADRE
SEQ ID NO: 18
aKXVAAWTLKAa
where “X” is cyclohexylalanine, and “a” is D-alanine
Linker-Coil29
SEQ ID NO: 19
SGSGQARILEADAEILRAYARILEAHAEILRAQ
PEPvlll-linker-Coil29
SEQ ID NO: 20
LEEKKGNYVVTDH SGSG QARILEADAEILRAYARILEAHAEILRAQ
PADRE-linker-Coil29
SEQ ID NO: 21
aKXVAAWTLKAa SGSG QARILEADAEILRAYARILEAHAEILRAQ
OVA epitope
SEQ ID NO: 22
ISQAVHAAHAEINEAGR
SEQ ID NO: 23
SIINFEKL
SEQ ID NO: 24
AAYGG
SEQ ID NO: 25
NPSHPLSGGGGS
SEQ ID NO: 26
(NPSHPLSGGGGS)₂K
SEQ ID NO: 27
((NPSHPLSGGGGS)₂K)₂
FP B-cell epitope
SEQ ID NO: 28
AVGIGAVFL
SEQ ID NO: 29
KSGSG
SEQ ID NO: 30
KKSGSG
SEQ ID NO: 31
(EAAAK)₂
Capping peptide with acetylated N-terminus (Ac) and amidated C-terminus (NH2)
SEQ ID NO: 32
Ac-QSRITEADSEITRAYSRITEAHSEITRAQ-NH2
Capping peptide with acetylated N-terminus (Ac) and amidated C-terminus (NH2)
SEQ ID NO: 33
Ac-QARILEADAEILRAYAEILEAHAEILRAQ-NH2
Capping peptide with acetylated N-terminus (Ac) and amidated C-terminus (NH2)
SEQ ID NO: 34
Ac-QARTLESDAETLRSYARTLESHAETLRSQ-NH2
Coil29 with acetylated N-terminus (Ac) and amidated C-terminus (NH2)
SEQ ID NO: 35
Ac-QARILEADAEILRAYARILEAHAEILRAQ-NH2
Self-assembling peptide with acetylated N-terminus (Ac) and
amidated C-terminus (NH2)
SEQ ID NO: 36
Ac-QAKILEADAEILKAYAKILEAHAEILKAQ-NH2
Coil23 with acetylated N-terminus (Ac) and amidated C-terminus (NH2)
SEQ ID NO: 37
Ac-ADAEILRAYARILEAHAEILRAQ-NH2
FP-Coil29
SEQ ID NO: 38
AVGIGAVFL-SGSG-(EAAAK-EAAAK)-SGSG-QARILEADAEILRAYARILEAHAEILRAQ-NH2
Alexa647-Coil29
SEQ ID NO: 39
AlexaFluor647-SGSG-(EAAAK-EAAAK)-SGSG-QARILEADAEILRAYARILEAHAEILRAQ-NH2
Biotin-FP
SEQ ID NO: 40
Biotin-SGSG-AVGIGAVFL
FP-Biotin
SEQ ID NO: 41
AVGIGAVFL-SGSG-(EAAAK-EAAAK)-SGSG-C-PEG-biotin
Capping peptide amino acid sequence
SEQ ID NO: 42
QSRITEADSEITRAYSRITEAHSEITRAQ
Capping peptide amino acid sequence
SEQ ID NO: 43
QARILEADAEILRAYAEILEAHAEILRAQ
Capping peptide amino acid sequence
SEQ ID NO: 44
QARTLESDAETLRSYARTLESHAETLRSQ
gonorrhea 2C7 epitope
SEQ ID NO: 45
IPVLDENGLFAP

1. A composition comprising:
(i) a nanofiber comprising:
a backbone comprising a plurality of self-assembling peptides, wherein each self-assembling peptide forms an alpha-helix, and wherein the self-assembling peptide comprises an amino acid sequence of bXXXb (SEQ ID NO: 1), wherein X is independently any amino acid, and b is independently any positively charged amino acid;
at least one glycomimetic peptide linked to the backbone; and
at least one antigen, wherein each antigen is individually linked to the backbone by a helical linker; or
(ii) a nanofiber comprising:
a backbone comprising a plurality of self-assembling peptides, wherein each self-assembling peptide forms an alpha-helix, and wherein the self-assembling peptide comprises an amino acid sequence of bXXXb (SEQ ID NO: 1), wherein X is independently any amino acid, and b is independently any positively charged amino acid; and
at least one antigen, wherein each antigen is individually linked to the backbone by a helical linker; or
(iii) a nanofiber comprising:
a backbone comprising a plurality of self-assembling peptides, wherein each self-assembling peptide forms an alpha-helix, and wherein the self-assembling peptide comprises an amino acid sequence of bXXXb (SEQ ID NO: 1), wherein X is independently any amino acid, and b is independently any positively charged amino acid; and
at least one glycomimetic peptide linked to the backbone; or
(iv) any combination of (i), (ii), and (iii).

2. The composition of claim 1, wherein the antigen comprises an epitope for a disease selected from HIV, inflammatory bowel disease, gonorrhea, or rheumatoid arthritis.

3. The composition of claim 1, wherein bXXXb (SEQ ID NO: 1) is RAYAR (SEQ ID NO: 2) or KAYAK (SEQ ID NO: 3).

4. The composition of claim 1, wherein the backbone comprises an amino acid sequence of ZnbXXXbZm (SEQ ID NO: 5), wherein b is independently any positively charged amino acid, Z is independently any amino acid, X is independently any amino acid, n is an integer from 0 to 20, and m is an integer from 0 to 20.

5. The composition of claim 1, wherein the backbone comprises an amino acid sequence selected from QARILEADAEILRAYARILEAHAEILRAQ (SEQ ID NO: 6), or QAKILEADAEILKAYAKILEAHAEILKAQ (SEQ ID NO: 7), or ADAEILRAYARILEAHAEILRAQ (SEQ ID NO: 8), or SEQ ID NO: 35, or SEQ ID NO: 36, or SEQ ID NO: 37.

6-9. (canceled)

10. The composition of claim 1, wherein each of the at least one glycomimetic peptide is capable of binding to a lectin.

11. The composition of claim 10, wherein the lectin comprises CD169.

12. The composition of claim 1, wherein the glycomimetic peptide is linked to the backbone by peptide linker.

13. The composition of claim 12, wherein the peptide linker comprises an amino acid sequence selected from SEQ ID NO: 9 (Gn wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), SEQ ID NO: 29 (KSGSG), SEQ ID NO: 30 (KKSGSG), and SEQ ID NO: 31 (EAAAK)₂.

14-16. (canceled)

17. The composition of claim 1, wherein the glycomimetic peptide comprises the amino acid sequence of SEQ ID NO: 25 [NPSHPLSGGGGS], SEQ ID NO: 26 [(NPSHPLSGGGGS)₂K], SEQ ID NO: 27 [((NPSHPLSGGGGS)₂K)₂], or a combination thereof.

18. (canceled)

19. The composition of claim 1, wherein the antigen comprises a FP B-cell epitope.

20. The composition of claim 19, wherein the FP B-cell epitope comprises the amino acid sequence of SEQ ID NO: 28 (AVGIGAVFL).

21. The composition of claim 1, wherein the antigen comprises a B cell epitope in TNF, or IL-17, or phosphorylcholine, or a complement C3dg, or a B cell epitope in complement C5a, or a peptide comprising the amino acid sequence of SEQ ID NO: 45.

22. (canceled)

23. The composition of claim 1, wherein the helical linker comprises a peptide comprising an amino acid sequence selected from SEQ ID NO: 9 (Gn wherein n is an integer from 1 to 10), SEQ ID NO: 10 (SGSG), SEQ ID NO: 11 (GSGS), SEQ ID NO: 12 (SSSS), SEQ ID NO: 13 (GGGS), SEQ ID NO: 14 (GGC), SEQ ID NO: 15 ((GGC)₈), SEQ ID NO: 16 ((G₄S)₃), and SEQ ID NO: 31 (EAAAK)₂.

24. The composition of claim 1, wherein the nanofiber further comprises one or more capping peptides.

25. The composition of claim 24, wherein the capping peptide comprises an amino acid sequence selected from SEQ ID NOs: 32, 33, 34, 42, 43, 44, or a combination thereof.

26. (canceled)

27. A method of immunizing a subject, the method comprising:
administering to the subject a therapeutically effective amount of the composition of claim 1.

28. The method of claim 27, further comprising administering an adjuvant, or a vaccine, or combination thereof, to the subject.

29. (canceled)

30. The method of claim 27, wherein the method further comprises administering a vaccine to the subject, and wherein the vaccine is retained in the lymph nodes for at least 6 hours, at least 12 hours, at least 36 hours, at least 48 hours after administration.

31. The method of claim 27, wherein the composition boosts germinal center reactions, thereby promoting a more robust immune response to the vaccine in the subject compared to a control,
or wherein the subject produces more IL-4 producing T cells after administration, compared to a control,
or wherein the subject produces more IFNγ producing T cells after administration, compared to a control,
or wherein the subject produces more antigen-binding antibodies or FP-binding antibodies after administration, compared to a control,
or a combination thereof.

32-35. (canceled)

36. The method of claim 30, wherein the vaccine comprises an HIV vaccine.

37-38. (canceled)

39. The method of claim 30, wherein the at least one nanofiber is administered prior to the vaccine, and wherein the composition of claim 1 primes the immune system of the subject prior to receiving the vaccine.

40-41. (canceled)
